(19) United States
(12) Patent Application Publication
(10) Pub. No.: US 2021/0145036 A1
(43) Pub. Date: May 20, 2021

(54) LIPOPHILIC ACTIVE AGENT INFUSED COMPOSITIONS WITH REDUCED FOOD EFFECT

(71) Applicant: POVIVA TEA, LLC, Reno, NV (US)

(72) Inventors: John Docherty, Port Perry (CA); Christopher Andrew Bunka, Kelowna (CA)

(21) Appl. No.: 17/047,470

(22) PCT Filed: Apr. 17, 2019
(86) PCT No.: PCT/US2019/027954

§ 371 Date: Oct. 14, 2020

Related U.S. Application Data
(60) Provisional application No. 62/659,059, filed on Apr. 17, 2018.

(51) Int. Cl. A23L 33/105; A23G 1/48; A23L 33/115; A23L 33/15; A23L 29/10; A61K 9/19; A23L 33/125; A61K 31/4468; A61K 31/485; A61K 31/5386; A61K 9/00; A23L 2/38

(52) U.S. Cl. CPC A23L 33/105; A23G 1/48; A23L 33/115; A23L 33/15; A23L 29/10; A23L 2/38; A23L 33/125; A61K 31/4468; A61K 31/485; A61K 31/5386; A61K 9/0053; A61K 9/19

(57) ABSTRACT

Aspects described herein relate to improved methods for infusing compositions with lipophilic active agents, particularly methods for reducing food effect in lipophilic active agent infused compositions, such as food and beverage compositions.

### CROSS-REFERENCE TO RELATED APPLICATIONS

This application is a PCT International Application which claims the benefit of U.S. Provisional Application No. 62/659,059, filed Apr. 17, 2018; which is incorporated herein by reference in its entirety.

### TECHNICAL FIELD

Aspects described herein relate to improved methods for infusing compositions with lipophilic active agents, particularly methods for reducing food effect in lipophilic active agent infused compositions, such as food and beverage compositions.

### BACKGROUND

Oral administration of drugs is frequently affected by food-drug interactions, a phenomenon known as “food effect”. As generally used, food effect is a broad term including all aspects of interactions of food on drug dissolution, absorption, distribution, metabolism, and elimination. In other words, the entire pharmacokinetic fate of the drug. Accordingly, food effects include, for example, changes in bioavailability, rate of on-set, duration of therapeutic effect, and incidence and seriousness of side effects.

The specific impact of food effect is therefore an issue of great importance during the development of a drug. If differences in the pharmacokinetic profile after administration in the fasted or fed state are too high, it can be difficult to define a safe and efficient therapeutic window for the drug. The bioavailability may vary unacceptably depending on a variety of factors, such as whether or not the drug has been taken with food or the kind of food with which the drug has been taken. Postprandial drug absorption may also be altered compared to the absorption from a fasting state in such a way that toxic effects can result.

In cases where food-drug interactions lead to an increase of drug absorption, the drug is recommended to be taken with food in order to be sufficiently absorbed and to exert its expected clinical effect. Such drug formulations are not ideal since drug absorption can vary depending on what kind of food has been consumed. If, by mistake, a patient forgets to take the drug formulation with food, clinical inefficiency could be the result of low absorption.

The pharmacokinetic profile of a drug is commonly described by the following parameters: Maximum plasma concentration (Cₘₐₓ), Time to maximum concentration (Tₘₐₓ), half-life (T_{1/2}), and Area under the curve (AUC). These parameters are to some extent interdependent, and are influenced in varying degrees by the outcome of interactions of food with drug dissolution, absorption, distribution, metabolism, and/or elimination. For example, for a certain drug, Cₘₐₓ might be mostly influenced by the rate of dissolution and absorption, while Tₘₐₓ might be mostly influenced by dissolution and distribution. For the same drug, T_{1/2} might be mostly influenced by metabolism and elimination, and AUC may be more or less influenced by all processes.

In most cases, changes in dissolution and absorption will have a significant impact on all parameters except perhaps on T_{1/2}. This implies that a formulation system, by which these two processes can be controlled and be made independent of food intake, will provide a more reliable and safer administration of the drug. Depending on the indication, the pharmacokinetic parameter that is most closely connected to therapeutic effect is either AUC, Cₘₐₓ, Tₘₐₓ or combinations thereof.

The effects of food on clinical pharmacokinetics have been reviewed (Singh (1999) Clinical Pharmacokinetics 37:213-255). In this review the effect of formulation type in bioequivalence studies is discussed, in which, under fasting and fed conditions, differences in pharmacokinetic parameters may be attributed to different formulation principles and excipient systems. The predominant role of pharmaceutical formulations applies not only for drug absorption, but also for hepatic first-pass metabolism, that a formulation exhibiting a good dissolution profile is less likely to be affected by a high-fat meal in spite of the lipophilicity of the drug. It is further mentioned that it is believed that the absorption of drugs solubilized by polyglycolised (polyethoxylated) glycerides is not affected by the presence or absence of food in the stomach.

While lipid based formulations have been shown to increase the bioavailability of a drug that is poorly absorbed from more conventional preparations, such improved preparations can still show a considerable food effect, or in some cases the food effect can become reversed. As discussed above, food effects commonly lead to unacceptable variability in the efficacy of a therapy, and can also constitute a hurdle for development of otherwise promising drug candidates.

There is therefore a strong need in the pharmaceutical industry today for a drug delivery system that can eliminate or reduce the food effect. Accordingly, there is a need for improved compositions and methods that can eliminate or reduce the food effect for the oral administration of lipophilic active agents to subjects in need thereof.

### SUMMARY

To address the foregoing problems, in whole or in part, and/or other problems that may have been observed by persons skilled in the art, the present disclosure provides compositions and methods as described by way of example as set forth below.

In one aspect, a process is provided for reducing food effect in a lipophilic active agent infused food product comprising the steps of:
(a) contacting a food product with an oil comprising a lipophilic active agent and a bioavailability enhancing agent; and
(b) dehydrating the food product;
   thereby producing a lipophilic active agent food product with reduced food effect; wherein the lipophilic active agent food product with reduced food effect comprises a therapeutically effective amount of the lipophilic active agent, and further wherein the bioavailability enhancing agent comprises an edible oil comprising long chain fatty acids and/or medium chain fatty acids; and further wherein:
(i) the lipophilic active agent is selected from the group consisting of cannabinoids, terpenes and terpenoids, non-steroidal anti-inflammatory drugs (NSAIDs), vitamins, nicotine, phosphodiesterase 5 (PDE5) inhibitors, Maca extract, hormones, fentanyl, buprenorphine, scopolamine, and antioxidants; and
(ii) the food product is selected from the group consisting of tea leaves, coffee beans, cocoa powder, meats, fish, fruits, vegetables, dairy products, legumes, pastas, breads, grains, seeds, nuts, spices, and herbs.
   In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In another aspect, processes are provided for reducing food effect in a lipophilic active agent infused beverage product comprising making lipophilic active agent infused tea leaves, coffee beans, or cocoa powder according to any of the processes described above; and further comprising the step of steeping the lipophilic active agent infused tea leaves, coffee beans, or cocoa powder in a liquid, thereby producing a lipophilic active agent infused beverage product with reduced food effect.

In another aspect, a process is provided for reducing food effect in a ready-to-drink beverage composition comprising a lipophilic active agent, obtainable by the steps of:
(a) contacting an emulsifier with an oil comprising a lipophilic active agent and a bioavailability enhancing agent, thereby producing a mixture comprising the emulsifier, the oil comprising the lipophilic active agent, and the bioavailability enhancing agent;
(b) dehydrating the mixture, thereby producing a dehydrated mixture comprising the emulsifier, the oil comprising the lipophilic active agent, and the bioavailability enhancing agent; and
(c) combining the dehydrated mixture with a ready-to-drink beverage composition, thereby producing a ready-to-drink beverage composition comprising the lipophilic active agent with reduced food effect;
   wherein:
(i) the bioavailability enhancing agent comprises an edible oil comprising long chain fatty acids and/or medium chain fatty acids;
(ii) the ready-to-drink beverage composition comprising a lipophilic active agent comprises a therapeutically effective amount of the lipophilic active agent; and
(iii) the lipophilic active agent is selected from the group consisting of cannabinoids, terpenes and terpenoids, non-steroidal anti-inflammatory drugs (NSAIDs), vitamins, a nicotine compound, phosphodiesterase 5 (PDE5) inhibitors, Maca extract, hormones, fentanyl, buprenorphine, scopolamine, and antioxidants.
   In particular aspects, the emulsifier is selected from the group consisting of inulin, gum arabic, modified starch, pectin, xanthan gum, gum ghatti, gum tragacanth, fenugreek gum, mesquite gum, mono-glycerides and di-glycerides of long chain fatty acids and/or medium chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, and combinations thereof. In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In another aspect, a method is provided for reducing food effect upon administration of a lipophilic active agent to a subject comprising administering a lipophilic active agent infused food product with reduced food effect to the subject, wherein the lipophilic active agent infused food product with reduced food effect is produced by the steps of:
(a) contacting a food product with an oil comprising a lipophilic active agent and a bioavailability enhancing agent; and
(b) dehydrating the food product;
   thereby producing a lipophilic active agent food product with reduced food effect; wherein the lipophilic active agent food product with reduced food effect comprises a therapeutically effective amount of the lipophilic active agent; wherein the bioavailability enhancing agent comprises an edible oil comprising long chain fatty acids and/or medium chain fatty acids; and further wherein:
(i) the lipophilic active agent is selected from the group consisting of cannabinoids, terpenes and terpenoids, non-steroidal anti-inflammatory drugs (NSAIDs), vitamins, a nicotine compound, phosphodiesterase 5 (PDE5) inhibitors, Maca extract, hormones, fentanyl, buprenorphine, scopolamine, and antioxidants; and
(ii) the food product is selected from the group consisting of the food product is selected from the group consisting of tea leaves, coffee beans, cocoa powder, meats, fish, fruits, vegetables, dairy products, legumes, pastas, breads, grains, seeds, nuts, spices, and herbs.
   In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In another aspect, methods are provided for reducing food effect upon administration of a lipophilic active agent to a subject comprising administering a lipophilic active agent infused beverage product with reduced food effect to the subject, wherein the lipophilic active agent infused beverage product with reduced food effect is produced by making lipophilic active agent infused tea leaves, coffee beans, or cocoa powder according to any of the processes described above; and further comprising the step of steeping the lipophilic active agent infused tea leaves, coffee beans, or cocoa powder in a liquid, thereby producing a lipophilic active agent infused beverage product with reduced food effect.

In another aspect, a method is provided for reducing food effect upon administration of a lipophilic active agent to a subject comprising administering a lipophilic active agent infused beverage product with reduced food effect to the subject, wherein the lipophilic active agent infused beverage product with reduced food effect is produced by the steps of making lipophilic active agent infused tea leaves, coffee beans, or cocoa powder according to any one of the processes described above; and further comprising the step of steeping the lipophilic active agent infused tea leaves, coffee beans, or cocoa powder in a liquid, thereby producing the lipophilic active agent infused beverage product with reduced food effect.

In another aspect, the bioavailability of the lipophilic active agent in a subject is at least 2 times, 5 times, or 10 times greater than the bioavailability of the lipophilic active agent in the subject in the absence of the edible oil comprising long chain fatty acids and/or medium chain fatty acids. In some aspects, the edible oil comprising long chain fatty acids and/or medium chain fatty acids is substantially free of omega-6 fatty acids (i.e., no more than trace amounts of omega-6 fatty acids). In some aspects, the long chain fatty acids and/or medium chain fatty acids are selected from the group consisting of oleic acid, undecanoic acid, valeric acid, heptanoic acid, pelargonic acid, capric acid, lauric acid, and eicosapentaenoic acid.

In some aspects, the lipophilic active agent is selected from the group consisting of: cannabinoids, terpenes and terpenoids, non-steroidal anti-inflammatory drugs (NSAIDs), vitamins, a nicotine compound, phosphodiesterase 5 (PDE5) inhibitors, Maca extract, hormones, fentanyl or an analog thereof, buprenorphine or an analog thereof, scopolamine or an analog thereof, and antioxidants. In some aspects, the cannabinoid is a psychoactive cannabinoid. In some aspects, the cannabinoid is a non-psychoactive cannabinoid. In some aspects, the NSAID is acetylsalicylic acid, ibuprophen, acetaminophen, diclofenac, indomethacin, piroxicam, or a COX inhibitor. In some aspects, the vitamin is vitamin A, D, E, or K. In some aspects, the PDE5 inhibitor is avanafil, lodenafil, mirodenafil, sildenafil, tadalafil, vardenafil, udenafil, acetildenafil, thiome-thisosildenafil, or analogs thereof. In some aspects, the hormone is an estrogen, an anti-estrogen, an androgen, an anti-androgen, or a progestin. In some aspects, the antioxidant is astaxanthin, Superoxide Dismusase, beta-carotene, selenium, lycopene, lutein, Coenzyme Q10, phytic acid, flavonoids, a polyphenol, a substituted 1,2-dihydroquinoline, ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, ice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

In some aspects, a method is provided for treating a condition comprising administering the disclosed compositions to a subject in need thereof, wherein the lipophilic active agent is a cannabinoid and the condition is selected from the group consisting of cardiac diseases such as heart disease, ischemic infarcts, and cardiometabolic disorders; neurological diseases such as Alzheimer's disease, Parkinson's disease, schizophrenia, and Human Immunodeficiency Virus (HIV) dementia; obesity; metabolic disorders such as insulin related deficiencies and lipid profiles, hepatic diseases, diabetes, and appetite disorders; cancer chemotherapy; benign prostatic hypertrophy; irritable bowel syndrome; biliary diseases; ovarian disorders; marijuana abuse; alcohol, opioid, nicotine, or cocaine addiction; and sexual dysfunction such as erectile dysfunction.

In some aspects, a method is provided for treating a condition comprising administering the disclosed compositions to a subject in need thereof, wherein the lipophilic active agent is a non-steroidal anti-inflammatory drug (NSAID), and wherein the condition is selected from the group consisting of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

In some aspects, a method is provided for treating a condition comprising administering the disclosed compositions to a subject in need thereof, wherein the lipophilic active agent is a vitamin, and wherein the condition is selected from the group consisting of a vitamin deficiency, vitamin malabsorption, and cystic fibrosis.

In some aspects, a method is provided for treating a condition comprising administering the disclosed compositions to a subject in need thereof, wherein the lipophilic active agent is a nicotine compound, and wherein the condition is selected from the group consisting of tobacco dependence/addiction, Parkinson's disease, ulcerative colitis, Alzheimer's disease, schizophrenia, Attention Deficit Hyperactivity Disorder (ADHD), Tourette's syndrome, ulcerous colitis, and post-smoking-cessation weight control.

In some aspects, a method is provided for treating a condition comprising administering the disclosed compositions to a subject in need thereof, wherein the lipophilic active agent is a phosphodiesterase 5 (PDE5) inhibitor, and wherein the condition is erectile dysfunction.

In some aspects, a method is provided for treating a condition comprising administering the disclosed compositions to a subject in need thereof, wherein the lipophilic active agent is Maca extract and wherein the condition is selected from the group consisting of inflammatory cytokine production, the effects of chronic inflammation, discomfort related to menstruation, the symptoms of menopause, the symptoms of andropause, the symptoms of HIV, the symptoms of anemia, discomfort related to chemotherapy, the symptoms of tuberculosis, the symptoms of osteoporosis, sexual dysfunction, and combinations thereof.

In some aspects, a method is provided for treating a condition comprising administering the disclosed compositions to a subject in need thereof, wherein the lipophilic active agent is a hormone and wherein the condition is a hormone deficiency.

In some aspects, a method is provided for treating a condition comprising administering the disclosed compositions to a subject in need thereof, wherein the lipophilic active agent is fentanyl and wherein the condition is pain.

In some aspects, a method is provided for treating a condition comprising administering the disclosed compositions to a subject in need thereof, wherein the lipophilic active agent is buprenorphine and wherein the condition is pain.

In some aspects, a method is provided for treating a condition comprising administering the disclosed compositions to a subject in need thereof, wherein the lipophilic active agent is scopolamine and wherein the condition is selected from the group consisting of nausea, vomiting, motion sickness, muscle spasms, and Parkinson-like conditions.

In some aspects, a method is provided for treating a condition comprising administering the disclosed compositions to a subject in need thereof, wherein the lipophilic active agent is an antioxidant and wherein the condition is oxidative stress in a mammalian cell.

In some aspects, a kit is provided comprising a lipophilic active agent infused composition and instructions for use thereof.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein at least one of the dosage forms exhibits a substantial positive food effect.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein the first and second dosage forms are the same and exhibit a substantial positive food effect.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein the first and second dosage form are the same and exhibit a substantial positive food effect, and the first and second doses are administered about 8 to about 16 hours apart.

In some aspects, a method is provided for administering a lipophilic active agent to a subject in need thereof, the method comprising: a) orally administering a first dose of a lipophilic active agent in a first extended release dosage form in the fed state, and b) orally administering a second dose of a lipophilic active agent in a second extended release dosage form in the fasting state, wherein the first and second dosage forms comprise different doses of a lipophilic active agent, the first dosage form exhibits a substantial positive food effect, and the first and second doses are administered about 8 to about 16 hours apart.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein: a) the first and second dosage form are the same and exhibit a substantial positive food effect; and b) the first dose is greater than the second dose, wherein the first dose is at least 1.2-fold greater than the second dose.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein: a) the first and second dosage form are the same and exhibit a substantial positive food effect; and b) the first dose is lower than the second dose, wherein the first dose is at least 1.2-fold lower than the second dose.

Other compositions, methods, features, and advantages of the invention will be or will become apparent to one with skill in the art upon examination of the following figures and detailed description. It is intended that all such additional compositions, methods, features, and advantages be included within this description, be within the scope of the invention, and be protected by the accompanying claims.

### BRIEF DESCRIPTION OF THE DRAWINGS

FIG. 1 shows Onset Performance based on 29 cases where DEHYDRATECH™ and Control versions were directly compared over day 2 and day 3 (with food). Non-heavy user cases=12; heavy user cases=17.

FIG. 2 shows Typical Edible Onset based on 36 cases with data collected on day 3. Non-heavy user cases=17; heavy user cases=19.

FIG. 3 shows Food Versus Onset based on cases where DEHYDRATECH™ version was tested with and without food (day 1 versus day 3). Non-heavy user cases=10; heavy user cases=12.

### DETAILED DESCRIPTION

The presently disclosed subject matter now will be described more fully hereinafter. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

In some embodiments, the compositions or methods comprise the specified components or steps. In some embodiments, the compositions or methods consist of the specified components or steps. In other embodiments, the compositions or methods consist essentially of the specified components or steps. As used herein, “consists essentially of” the specified components or steps means that the composition includes at least the specified components or steps, and may also include other components or steps that do not materially affect the basic and novel characteristics of the invention.

Aspects described herein relate to improved methods for infusing compositions with lipophilic active agents, particularly methods for reducing food effect in lipophilic active agent infused compositions, such as food and beverage compositions.

### I. Methods for Reducing Food Effect in Lipophilic Active Agent Infused Compositions

In one aspect, a process is provided for reducing food effect in a lipophilic active agent infused food product comprising the steps of:

(a) contacting a food product with an oil comprising a lipophilic active agent and a bioavailability enhancing agent; and

(b) dehydrating the food product;

thereby producing a lipophilic active agent food product with reduced food effect; wherein the lipophilic active agent food product with reduced food effect comprises a therapeutically effective amount of the lipophilic active agent, and further wherein the bioavailability enhancing agent comprises an edible oil comprising long chain fatty acids and/or medium chain fatty acids; and further wherein:
(i) the lipophilic active agent is selected from the group consisting of cannabinoids, terpenes and terpenoids, non-steroidal anti-inflammatory drugs (NSAIDs), vitamins, a nicotine compound, phosphodiesterase 5 (PDE5) inhibitors, Maca extract, hormones, fentanyl, buprenorphine, scopolamine, and antioxidants; and
(ii) the food product is selected from the group consisting of tea leaves, coffee beans, cocoa powder, meats, fish, fruits, vegetables, dairy products, legumes, pastas, breads, grains, seeds, nuts, spices, and herbs.
   In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme. In other aspects, the bioavailability of the lipophilic active agent in a subject is at least 1.5 times greater than the bioavailability of the lipophilic active agent in the subject in the absence of the edible oil comprising long chain fatty acids and/or medium chain fatty acids, particularly at least 3 times greater, and more particularly at least 4.5 times greater. In other aspects, the edible oil comprising long chain fatty acids and/or medium chain fatty acids is substantially free of omega-6 fatty acids.

In another aspect, processes are provided for reducing food effect in a lipophilic active agent infused beverage product comprising making lipophilic active agent infused tea leaves, coffee beans, or cocoa powder according to any of the processes described above; and further comprising the step of steeping the lipophilic active agent infused tea leaves, coffee beans, or cocoa powder in a liquid, thereby producing a lipophilic active agent infused beverage product with reduced food effect.

In another aspect, a process is provided for reducing food effect in a ready-to-drink beverage composition comprising a lipophilic active agent, obtainable by the steps of:
(a) contacting an emulsifier with an oil comprising a lipophilic active agent and a bioavailability enhancing agent, thereby producing a mixture comprising the emulsifier, the oil comprising the lipophilic active agent, and the bioavailability enhancing agent;
(b) dehydrating the mixture, thereby producing a dehydrated mixture comprising the emulsifier, the oil comprising the lipophilic active agent, and the bioavailability enhancing agent; and
(c) combining the dehydrated mixture with a ready-to-drink beverage composition, thereby producing a ready-to-drink beverage composition comprising the lipophilic active agent with reduced food effect;
   wherein:
(i) the bioavailability enhancing agent comprises an edible oil comprising long chain fatty acids and/or medium chain fatty acids;
(ii) the ready-to-drink beverage composition comprising a lipophilic active agent comprises a therapeutically effective amount of the lipophilic active agent; and
(iii) the lipophilic active agent is selected from the group consisting of cannabinoids, terpenes and terpenoids, non-steroidal anti-inflammatory drugs (NSAIDs), vitamins, a nicotine compound, phosphodiesterase 5 (PDE5) inhibitors, Maca extract, hormones, fentanyl, buprenorphine, scopolamine, and antioxidants.
   In particular aspects, the emulsifier is selected from the group consisting of inulin, gum arabic, modified starch, pectin, xanthan gum, gum ghatti, gum tragacanth, fenugreek gum, mesquite gum, mono-glycerides and di-glycerides of long chain fatty acids and/or medium chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, and combinations thereof. In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In another aspect, a method is provided for reducing food effect upon administration of a lipophilic active agent to a subject comprising administering a lipophilic active agent infused food product with reduced food effect to the subject, wherein the lipophilic active agent infused food product with reduced food effect is produced by the steps of:
(a) contacting a food product with an oil comprising a lipophilic active agent and a bioavailability enhancing agent; and
(b) dehydrating the food product;
   thereby producing a lipophilic active agent food product with reduced food effect; wherein the lipophilic active agent food product with reduced food effect comprises a therapeutically effective amount of the lipophilic active agent; wherein the bioavailability enhancing agent comprises an edible oil comprising long chain fatty acids and/or medium chain fatty acids; and further wherein:
(i) the lipophilic active agent is selected from the group consisting of cannabinoids, terpenes and terpenoids, non-steroidal anti-inflammatory drugs (NSAIDs), vitamins, a nicotine compound, phosphodiesterase 5 (PDE5) inhibitors, Maca extract, hormones, fentanyl, buprenorphine, scopolamine, and antioxidants; and
(ii) the food product is selected from the group consisting of the food product is selected from the group consisting of tea leaves, coffee beans, cocoa powder, meats, fish, fruits, vegetables, dairy products, legumes, pastas, breads, grains, seeds, nuts, spices, and herbs.
   In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In another aspect, methods are provided for reducing food effect upon administration of a lipophilic active agent to a subject comprising administering a lipophilic active agent infused beverage product with reduced food effect to the subject, wherein the lipophilic active agent infused beverage product with reduced food effect is produced by making lipophilic active agent infused tea leaves, coffee beans, or cocoa powder according to any of the processes described above; and further comprising the step of steeping the lipophilic active agent infused tea leaves, coffee beans, or cocoa powder in a liquid, thereby producing a lipophilic active agent infused beverage product with reduced food effect.

In another aspect, a method is provided for reducing food effect upon administration of a lipophilic active agent to a subject comprising administering a lipophilic active agent infused beverage product with reduced food effect to the subject, wherein the lipophilic active agent infused beverage product with reduced food effect is produced by the steps of making lipophilic active agent infused tea leaves, coffee beans, or cocoa powder according to any one of the processes described above; and further comprising the step of steeping the lipophilic active agent infused tea leaves, coffee beans, or cocoa powder in a liquid, thereby producing the lipophilic active agent infused beverage product with reduced food effect.

In another aspect, the bioavailability of the lipophilic active agent in a subject is at least 2 times, 5 times, or 10 times greater than the bioavailability of the lipophilic active agent in the subject in the absence of the edible oil comprising long chain fatty acids and/or medium chain fatty acids. In some aspects, the edible oil comprising long chain fatty acids and/or medium chain fatty acids is substantially free of omega-6 fatty acids. In some aspects, the long chain fatty acids and/or medium chain fatty acids are selected from the group consisting of oleic acid, undecanoic acid, valeric acid, heptanoic acid, pelargonic acid, capric acid, lauric acid, and eicosapentaenoic acid. In some aspects, the dosage form of the invention exhibits a substantial positive food effect, whereby it provides an increased Cₘₐₓ and AUC when administered orally in the fed state as compared to the fasting state. The positive food effect can be used to alter the dosing regimen of the dosage form. In some embodiments, the invention provides a method of administering a lipophilic active agent to a subject in need thereof, the method comprising: a) orally administering a first dose of a composition of the invention in the fed state, and b) orally administering a second dose of a composition of the invention in the fasting state.

In some embodiments, the invention provides a method of administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein at least one of the dosage forms exhibits a substantial positive food effect.

In some embodiments, the invention provides a method of administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein the first and second dosage forms are the same and exhibit a substantial positive food effect.

In some embodiments, the invention provides a method of administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein the first and second dosage form are the same and exhibit a substantial positive food effect, and the first and second doses are administered about 8 to about 16 hours apart.

In some embodiments, the invention provides a method of administering a lipophilic active agent to a subject in need thereof, the method comprising: a) orally administering a first dose of a lipophilic active agent in a first extended release dosage form in the fed state, and b) orally administering a second dose of a lipophilic active agent in a second extended release dosage form in the fasting state, wherein the first and second dosage forms comprise different doses of a lipophilic active agent, the first dosage form exhibits a substantial positive food effect, and the first and second doses are administered about 8 to about 16 hours apart.

In some embodiments, the invention provides a method of administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein: a) the first and second dosage form are the same and exhibit a substantial positive food effect; and b) the first dose is greater than the second dose. In some embodiments, the first dose is at least 1.2-fold, at least 1.5-fold, at least 1.75-fold or at least 2-fold greater than the second dose.

In some embodiments, the invention provides a method of administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein: a) the first and second dosage form are the same and exhibit a substantial positive food effect; and b) the first dose is lower than the second dose. In some embodiments, the first dose is at least 1.2-fold, at least 1.5-fold, at least 1.75-fold or at least 2-fold lower than the second dose.

In some embodiments, the different daily dose is less than 50 by wt % or mole %, on the basis of a lipophilic active agent, of the current daily dose. In some embodiments, the second method provides about the same clinical benefit as or provides an improved clinical benefit over the first (current) method. In some embodiments, the second method employs less than 50% of the dose of a lipophilic active agent as compared to the first (current) method.

In some embodiments, the subject's current method of treatment is ceased before initiating the second method of treatment. In some embodiments, the subject's current method of treatment and the second method of treatment according to the invention overlap.

The food effect can also be used to advantage to further control the absorption of a lipophilic active agent. For example, a subject can be orally administered a dose under fasting conditions and later a dose under fed conditions in a single day, or vice versa. For example, a subject could be administered the first dose with food and a second dose about 8 to 16 hours later. Fasting conditions are established by abstaining from consumption of food for at least 2, at least 3 or at least 4 hours before administration of a dose. The first and second doses and/or dosage forms can be the same or different. One or both dosage forms will exhibit a substantial positive food effect.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein at least one of the dosage forms exhibits a substantial positive food effect.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein the first and second dosage forms are the same and exhibit a substantial positive food effect.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein the first and second dosage form are the same and exhibit a substantial positive food effect, and the first and second doses are administered about 8 to about 16 hours apart.

In some aspects, a method is provided for administering a lipophilic active agent to a subject in need thereof, the method comprising: a) orally administering a first dose of a lipophilic active agent in a first extended release dosage form in the fed state, and b) orally administering a second dose of a lipophilic active agent in a second extended release dosage form in the fasting state, wherein the first and second dosage forms comprise different doses of a lipophilic active agent, the first dosage form exhibits a substantial positive food effect, and the first and second doses are administered about 8 to about 16 hours apart.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein: a) the first and second dosage form are the same and exhibit a substantial positive food effect; and b) the first dose is greater than the second dose, wherein the first dose is at least 1.2-fold greater than the second dose.

In some aspects, a method is provided for administering a lipophilic active agent to a subject in need thereof, the method comprising: a) orally administering a first dose of a lipophilic active agent in a composition in the fed state, and b) orally administering a second dose of a lipophilic active agent in a composition in the fasting state, wherein: a) the first and second dosage form are the same and exhibit a substantial positive food effect; and b) the first dose is lower than the second dose, wherein the first dose is at least 1.2-fold lower than the second dose.

A. Cannabinoids

Cannabis sativa L. is one of the most widely used plants for both recreational and medicinal purposes. Over 500 natural constituents have been isolated and identified from C. sativa covering several chemical classes (Ahmed et al. (2008) J. Nat. Prod. 71:536-542; Ahmed et al. (2008) Tetrahedron Lett. 49:6050-6053; ElSohly & Slade (2005) Life Sci. 78:539-548; Radwan et al. (2009) J. Nat. Prod. 72:906-911; Radwan et al. (2008) Planta Medial. 74:267-272; Radwan et al. (2008) J. Nat. Prod. 69:2627-2633; Ross et al. (1995) Zagazig J. Pharm. Sci. 4:1-10; Turner et al. (1980) J. Nat. Prod. 43:169-170). Cannabinoids belong to the chemical class of terpenophenolics, of which at least 85 have been uniquely identified in cannabis (Borgelt et al. (2013) Pharmacotherapy 33:195-209).

Cannabinoids are ligands to cannabinoid receptors (CB₁, CB₂) found in the human body (Pertwee (1997) Pharmacol. Ther. 74:129-180). The cannabinoids are usually divided into the following groups: classical cannabinoids; non-classical cannabinoids; aminoalkylindole-derivatives; and eicosanoids (Pertwee (1997) Pharmacol. Ther. 74:129-180). Classical cannabinoids are those that have been isolated from C. sativa L. or their synthetic analogs. Non-classical cannabinoids are bi- or tri-cyclic analogs of tetrahydrocannabinol (THC) (without the pyran ring). Aminoalkylindoles and eicosanoids are substantially different in structure compared to classical and non-classical cannabinoids. The most common natural plant cannabinoids (phytocannabinoids) are cannabidiol (CBD), cannabigerol (CBG), cannabichromene (CBC), and cannabinol (CBN). The most psychoactive cannabinoid is Δ⁹-THC.

In recent years, marijuana and its components have been reported in scientific literature to counter the symptoms of a broad range of conditions including but not limited to multiple sclerosis and other forms of muscular spasm; movement disorders; pain, including migraine headache; glaucoma; asthma; inflammation; insomnia; and high blood pressure. There may also be utility for cannabinoids as anxiolytics, anti-convulsives, anti-depressants, anti-psychotics, anti-cancer agents, as well as appetite stimulants. Pharmacological and toxicological studies of cannabinoids have largely been focused on a synthetic analog of Δ⁹-THC (commercially available under the generic name Dronabinol). In 1985, Dronabinol was approved by the FDA for the treatment of chemotherapy associated nausea and vomiting, and later for AIDS-associated wasting and anorexia.

Therapeutic use of cannabinoids has been hampered by the psychoactive properties of some compounds (e.g., Dronabinol) as well as their low bioavailability when administered orally. Bioavailability refers to the extent and rate at which the active moiety (drug or metabolite) enters systemic circulation, thereby accessing the site of action. The low bioavailability of orally ingested cannabinoids (from about 6% to 20%; Adams & Martin (1996) Addiction 91: 1585-614; Agurell et al. (1986) Pharmacol. Rev. 38: 21-43; Grotenhermen (2003) Clin. Pharmacokinet. 42: 327-60) has been attributed to their poor dissolution properties and extensive first pass metabolism.

Cannabinoids are a heteromorphic group of chemicals which directly or indirectly activate the body's cannabinoid receptors. There are three main types of cannabinoids: herbal cannabinoids that occur uniquely in the cannabis plant, synthetic cannabinoids that are manufactured, and endogenous cannabinoids that are produced in vivo. Herbal cannabinoids are nearly insoluble in water but soluble in lipids, alcohol, and non-polar organic solvents. These natural cannabinoids are concentrated in a viscous resin that is produced in glandular structures known as trichomes. In addition to cannabinoids, the resin is rich in terpenes, which are largely responsible for the odor of the cannabis plant.

The identification of Δ⁹-tetrahydrocannabinol (THC) as a major psychoactive drug and its chemical synthesis in 1964 opened a new era of synthetic cannabinoids as pharmacological agents. Cannabinoid research has increased tremendously in recent years since the discovery of cannabinoid receptors and the endogenous ligands for these receptors. The receptors include CB1, predominantly expressed in the brain, and CB2, primarily found on the cells of the immune system. Cannabinoid receptors belong to a superfamily of G-protein-coupled receptors. They are single polypeptides with seven transmembrane α-helices, and have an extracellular, glycosylated N-terminus and intracellular C-terminus. Both CB1 and CB2 cannabinoid receptors are linked to G1/0-proteins. In addition to these receptors, endogenous ligands for these receptors capable of mimicking the pharmacological actions of THC have also been discovered. Such ligands were designated endocannabinoids and included anandamide and 2-arachidonoyl glycerol (2-AG). Anandamide is produced in the brain and peripheral immune tissues such as the spleen.

Unlike THC, which exerts its action by binding to CB1 and CB2, cannabidiol does not bind to these receptors and hence has no psychotropic activity. Instead, cannabidiol indirectly stimulates endogenous cannabinoid signaling by suppressing the enzyme that breaks down anandamide (fatty acid amide hydroxylase, “FAAH”). Cannabidiol also stimulates the release of 2-AG. Cannabidiol has been reported to have immunomodulating and anti-inflammatory properties, to exhibit anticonvulsive, anti-anxiety, and antipsychotic activity, and to function as an efficient neuroprotective antioxidant.

Cannabinoids in cannabis are often inhaled via smoking, but may also be ingested. Smoked or inhaled cannabinoids have reported bioavailabilities ranging from 2-56%, with an average of about 30% (Huestis (2007) Chem. Biodivers. 4:1770-1804; McGilveray (2005) Pain Res. Manag. 10 Suppl. A:15A-22A). This variability is mainly due to differences in smoking dynamics. Cannabinoids that are absorbed through the mucous membranes in the mouth (buccomucosal application) have bioavailabilities of around 13% (Karschner et al. (2011) Clin. Chem. 57:66-75). By contrast, when cannabinoids are ingested, bioavailability is typically reduced to about 6% (Karschner et al. (2011) Clin. Chem. 57:66-75).

Accordingly, in other aspects, within the compositions and methods of the present invention, the lipophilic active agent is a cannabinoid.

In particular aspects, at least one cannabinoid within the compositions and methods of the present invention is selected from the group consisting of:

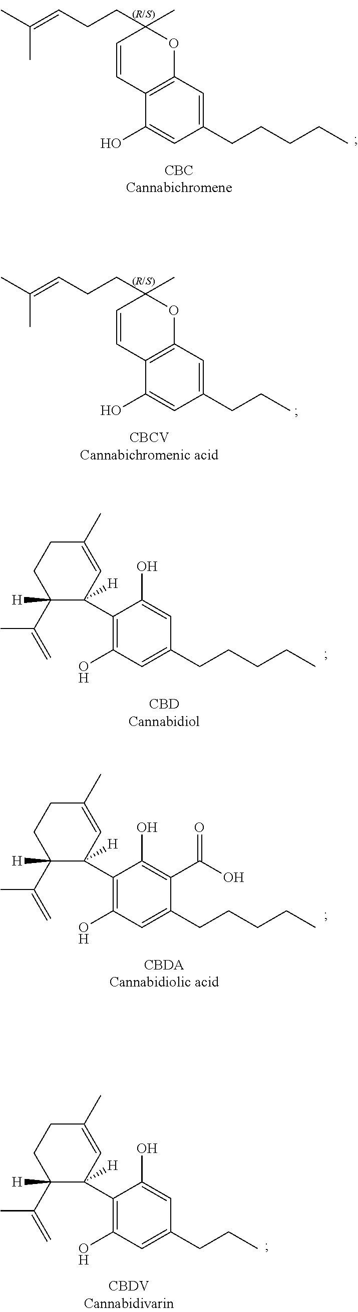

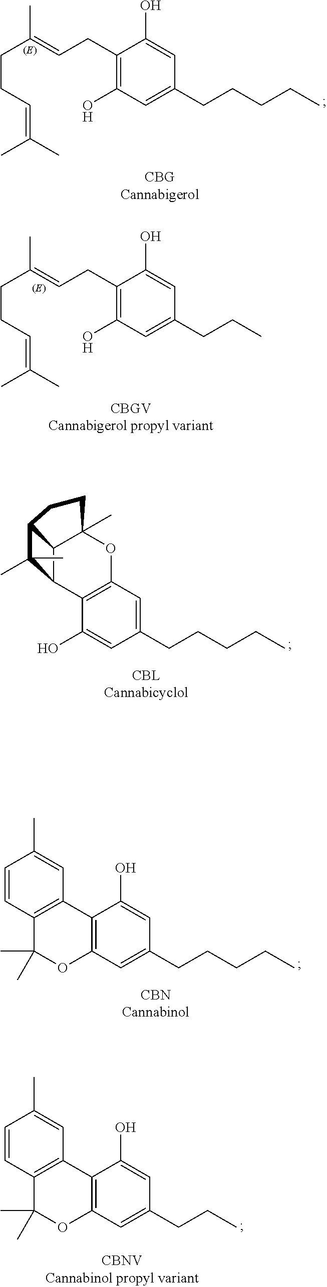

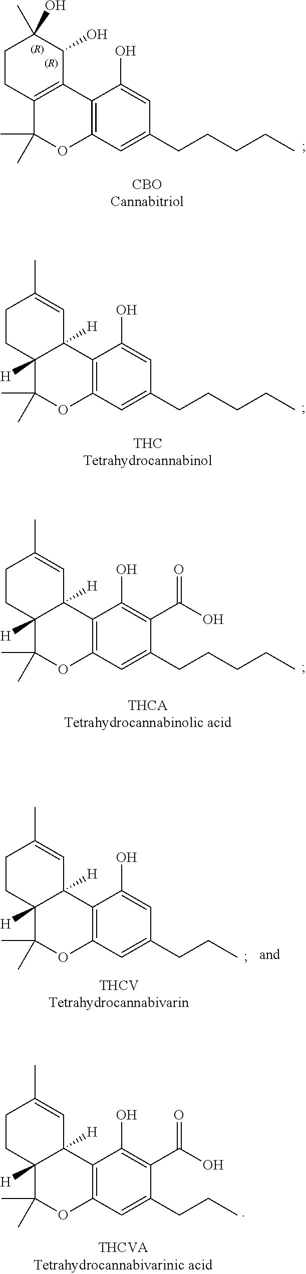

In particular aspects, at least one cannabinoid within the compositions and methods of the present invention is a non-psychoactive cannabinoid such as cannabidiol. In some particularly disclosed aspects, the cannabinoid is selected from the group consisting of:

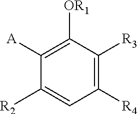

where A is aryl, and particularly

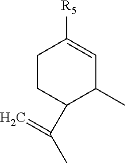

but not a pinene such as:

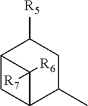

and the R₁-R₅ groups are each independently selected from the groups of hydrogen, lower substituted or unsubstituted alkyl, substituted or unsubstituted carboxyl, substituted or unsubstituted alkoxy, substituted or unsubstituted alcohol, and substituted or unsubstituted ethers, and R₆-R₇ are H or methyl. In particular aspects, there are no nitrogens in the rings, and/or no amino substitutions on the rings.

In other aspects, the cannabinoid is selected from the group consisting of:

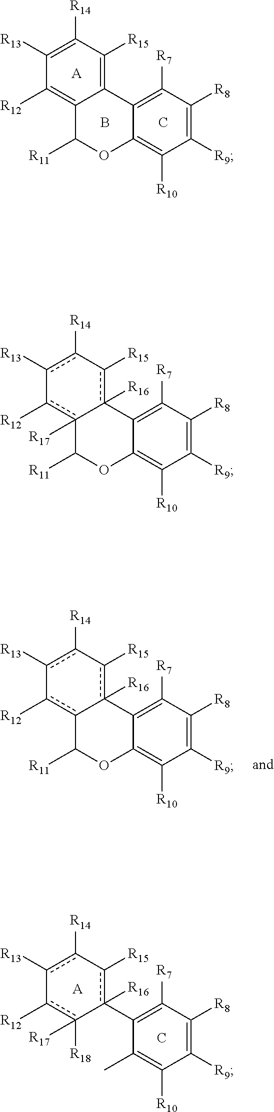

where there can be 0 to 3 double bonds on the A ring, as indicated by the optional double bonds indicated by dashed lines on the A ring. The C ring is aromatic, and the B ring can be a pyran. Particular aspects are dibenzo pyrans and cyclohexenyl benzenediols. Particular aspects of the cannabinoids of the present invention may also be highly lipid soluble, and in particular aspects can be dissolved in an aqueous solution only sparingly (for example 10 mg/ml or less). The octanol/water partition ratio at neutral pH in useful aspects is 5000 or greater, for example 6000 or greater. This high lipid solubility enhances penetration of the drug into the central nervous system (CNS), as reflected by its volume of distribution (V_{d}) of 1.5 L/kg or more, for example 3.5 L/kg, 7 L/kg, or ideally 10 L/kg or more, for example at least 20 L/kg. Particular aspects may also be highly water soluble derivatives that are able to penetrate the CNS, for example carboxyl derivatives.

R₇₋₁₈ are independently selected from the group of H, substituted or unsubstituted alkyl, especially lower alkyl, for example unsubstituted C₁-C₃ alkyl, hydroxyl, alkoxy, especially lower alkoxy such as methoxy or ethoxy, substituted or unsubstituted alcohol, and unsubstituted or substituted carboxyl, for example COOH or COCH₃. In other aspects R₇₋₁₈ can also be substituted or unsubstituted amino, and halogen.

In particular aspects, at least one cannabinoid within the compositions and methods of the present invention is a non-psychoactive cannabinoid, meaning that the cannabinoid has substantially no psychoactive activity mediated by the cannabinoid receptor (for example an IC₅₀ at the cannabinoid receptor of greater than or equal to 300 nM, for example greater than 1 μM and a Kᵢ greater than 250 nM, especially 500-1000 nM, for example greater than 1000 nM).

In other particular aspects, the cannabinoids within the compositions and methods of the present invention are selected from the group consisting of:

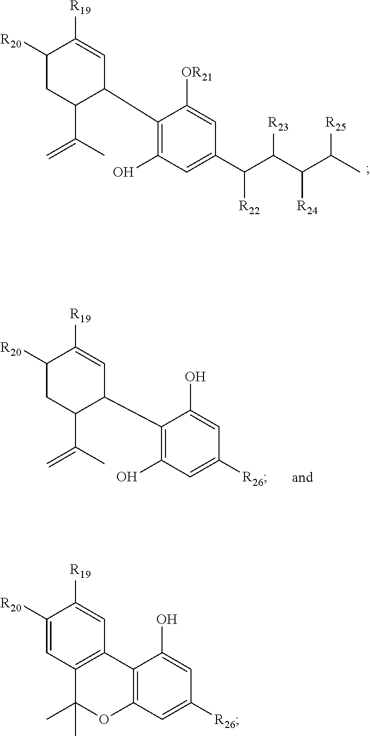

where R₁₉ is substituted or unsubstituted alkyl, such as lower alkyl (for example methyl), lower alcohol (such as methyl alcohol) or carboxyl (such as carboxylic acid) and oxygen (as in ═O); R₂₀ is hydrogen or hydroxy; R₂₁ is hydrogen, hydroxy, or methoxy; R₂₂ is hydrogen or hydroxy; R₂₃ is hydrogen or hydroxy; R₂₄ is hydrogen or hydroxy; R₂₅ is hydrogen or hydroxy; and R₂₆ is substituted or unsubstituted alkyl (for example n-methyl alkyl), substituted or unsubstituted alcohol, or substituted or unsubstituted carboxy.

In other particular aspects, the cannabinoids within the compositions and methods of the present invention are selected from the group consisting of:

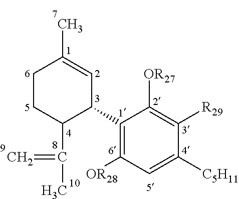

wherein numbering conventions for each of the ring positions are shown, and R₂₇, R₂₈ and R₂₉ are independently selected from the group consisting of H, unsubstituted lower alkyl such as CH₃, and carboxyl such as COCH₃. Particular examples of nonpsychoactive cannabinoids that fall within this definition are cannabidiol and

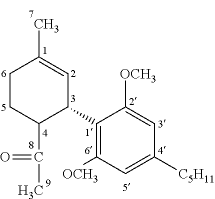

and other structural analogs of cannabidiol.

In other particular aspects, the cannabinoids within the compositions and methods of the present invention are selected from the group consisting of:

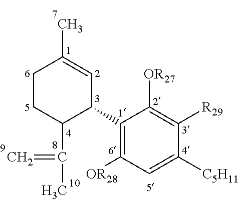

wherein R₂₇, R₂₈ and R₂₉ are independently selected from the group consisting of H, lower alkyl such as CH₃, and carboxyl such as COCH₃, and particularly wherein:

a) R₂₇═R₂₈=R₂₉═H

b) R₂₇═R₂₉═H; R₂₈═CH₃

c) R₂₇═R₂₈═CH₃; R₂₉═H

d) R₂₇═R₂₈═COCH₃; R₂₉═H

e) R₂₇═H; R₂₈═R₂₉═COCH₃

When R₂₇═R₂₈=R₂₉═H, then the compound is cannabidiol (CBD). When R₂₇═R₂₉═H and R₂₈═CH₃, the compound is CBD monomethyl ether. When R₂₇═R₂₈═CH₃ and R₂₉═H, the compound is CBD dimethyl ether. When R₂₇═R₂₈═COCH₃ and R₂₉═H, the compound is CBD diacetate. When R₂₇═H and R₂₈═R₂₉═COCH₃, the compound is CBD monoacetate.

B. Terpenes and Terpenoids

Terpenes are a diverse group of organic hydrocarbons derived from 5-carbon isoprene units and are produced by a wide variety of plants. Terpenoids are terpenes which have been chemically modified to add functional groups including heteroatoms. Terpenes and terpenoids are important building blocks for hormones, vitamins, pigments, steroids, resins, and essential oils. Terpenes are naturally present in cannabis; however, they can be removed during the extraction process. Terpenes and terpenoids have various pharmaceutical (pharmacodynamic) effects and can be selected for the desired pharmaceutical activities.

In one embodiment, the terpene/terpenoid includes limonene. Limonene is a colorless liquid hydrocarbon classified as a cyclic terpene. The more common D-isomer possesses a strong smell of oranges and a bitter taste. It is used in chemical synthesis as a precursor to carvone and as a solvent in cleaning products. Limonene is a chiral molecule. Biological sources produce one enantiomer—the principal industrial source—citrus fruit, contains D-limonene ((+)-limonene), which is the (R)-enantiomer (CAS number 5989-27-5, EINECS number 227-813-5). Racemic limonene is known as dipentene. Its IUPAC name is 1-methyl-4-(1-methylethenyl)-cyclohexene. It is also known as 4-isopropenyl-1-methylcyclohexenep-Menth-1,8-dieneRacemic: DL-limonene; dipentene.

Limonene has a history of use in medicine, food and perfume. It has very low toxicity, and humans are rarely allergic to it. Limonene is used as a treatment for gastric reflux and as an anti-fungal agent. Its ability to permeate proteins makes it a useful treatment for toenail fungus. Limonene is also used for treating depression and anxiety. Limonene is reported to assist in the absorption of other terpenoids and chemicals through the skin, mucous membranes and digestive tract. Limonene has immunostimulant properties. Limonene is also used as botanical insecticide

The principle metabolites of limonene are (+)- and (−)-trans-carveol, a product of 6-hydroxylation) and (+)- and (−)-perillyl alcohol, a product of 7-hydroxylation by CYP2C9 and CYP2C19 cytochromes in human liver microsomes. The enantiomers of perillyl alcohol have been researched for possible pharmacological possibilities as dietary chemotherapeutic agents. They are considered novel therapeutic options in some CNS neoplasms and other solid tumors, especially for treatment of gliomas. The cytotoxic activities of perillyl alcohol and limonene metabolites are likely due to their antiangiogenic properties, hyperthermia inducing effects, negative apoptosis regulation and effect on Ras pathways.

In another embodiment, the terpene/terpenoid includes linalool. Linalool is a naturally occurring terpene alcohol chemical found in many flowers and spice plants with many commercial applications, the majority of which are based on its pleasant scent (floral and slightly spicy). It is also known as β-linalool, linalyl alcohol, linaloyl oxide, p-linalool, allo-ocimenol, and 3,7-dimethyl-1,6-octadien-3-ol. Its IUPAC name is 3,7-dimethylocta-1,6-dien-3-ol.

More than 200 species of plants produce linalool, mainly in the families Lamiaceae, Lauraceae and Rutaceae. It has also been found in some fungi. Linalool has been used for thousands of years as a sleep aid. Linalool is an important precursor in the formation of Vitamin E. It has a history of use in the treatment of both psychosis and anxiety, and as an anti-epileptic agent. It also provides analgesic pain relief. Its vapors have been shown to be an effective insecticide against fleas, fruit flies and cockroaches. Linalool is used as a scent in an estimated 60-80% of perfumed hygiene products and cleaning agents including soaps, detergents, shampoos and lotions.

In another embodiment, the terpene/terpenoid includes myrcene. Myrcene, or β-myrcene, is an olefinic natural organic compound. It is classified as a hydrocarbon, more precisely as a monoterpene. Terpenes are dimers of isoprene, and myrcene is one of the most important. Myrcene is a component of the essential oil of several plants including bay, cannabis, ylang-ylang, wild thyme, mango, parsley and hops. Myrcene is produced mainly semi-synthetically from myrcia, from which it gets its name. Myrcene is a key intermediate in the production of several fragrances. α-Myrcene is the name for the structural isomer 2-methyl-6-methylene-1,7-octadiene, which is not found in nature and is little used. Its IUPAC name is 7-methyl-3-methylene-1,6-octadiene.

Myrcene has an analgesic effect and is likely to be responsible for the medicinal properties of lemon grass tea. It has anti-inflammatory properties through Prostaglandin E2. The analgesic action can be blocked by naloxone or yohimbine in mice, which suggests mediation by alpha 2-adrenoceptor stimulated release of endogenous opioids. β-Myrcene is reported to have anti-inflammatory properties, and is used to treat spasms, sleep disorders and pain. Myrcene appears to lower resistance across the blood to brain barrier, allowing itself and many other chemicals to cross the barrier more effectively.

In another embodiment, the terpene/terpenoid includes α-Pinene. α-Pinene is one of the primary monoterpenes that is physiologically critical in both plants and animals. It is an alkene and it contains a reactive four-membered ring. α-Pinene tends to react with other chemicals, forming a variety of other terpenes including D-limonene and other compounds. α-Pinene has been used for centuries as a bronchodilator in the treatment of asthma. It is highly bioavailable with 60% human pulmonary uptake with rapid metabolism. α-Pinene is an anti-inflammatory via PGE1, and appears to be a broad-spectrum antibiotic. It acts as an acetylcholinesterase inhibitor, aiding memory. Products of α-pinene which have been identified include pinonaldehyde, norpinonaldehyde, pinic acid, pinonic acid, and pinalic acid.

Pinene is found in conifer, pine and orange. α-Pinene is a major constituent in turpentine. Its IUPAC name is (1S,5S)-2,6,6-Trimethylbicyclo[3.1.1]hept-2-ene ((−)-α-Pinene).

In another embodiment, the terpene/terpenoid includes β-Pinene. β-Pinene is one of the most abundant compounds released by trees. It is one of the two isomers of pinene, the other being α-pinene. It is a common monoterpene, and if oxidized in air, the allylic products of the pinocarveol and myrtenol family prevail. Its IUPAC name is 6,6-dimethyl-2-methylenebicyclo[3.1.1]heptane and is also known as 2(10)-Pinene; Nopinene; Pseudopinene. It is found in cumin, lemon, pine and other plants.

In another embodiment, the terpene/terpenoid includes caryophyllene, also known as β-caryophyllene. Caryophyllene is a natural bicyclic sesquiterpene that is a constituent of many essential oils, including clove, cannabis, rosemary and hops. It is usually found as a mixture with isocaryophyllene (the cis double bond isomer) and α-humulene, a ring-opened isomer. Caryophyllene is notable for having a rare cyclobutane ring. Its IUPAC name is 4,11,11-trimethyl-8-methylene-bicyclo[7.2.0]undec-4-ene.

Caryophyllene is known to be one of the compounds that contribute to the spiciness of black pepper. In a study conducted by the Swiss Federal Institute of Technology, β-caryophyllene was shown to be selective agonist of cannabinoid receptor type-2 (CB2) and to exert significant cannabimimetic, anti-inflammatory effects in mice. Anti-nociceptive, neuroprotective, anxiolytic, antidepressant and anti-alcoholic activity have been tied to caryophyllene. Because β-caryophyllene is an FDA approved food additive, it is considered the first dietary cannabinoid.

In another embodiment, the terpene/terpenoid includes citral. Citral, or 3,7-dimethyl-2,6-octadienal or lemonal, is either a pair, or a mixture of terpenoids with the molecular formula C₁₀H₁₆O. The two compounds are double bond isomers. The E-isomer is known as geranial or citral A. The Z-isomer is known as neral or citral B. Its IUPAC name is 3,7-dimethylocta-2,6-dienal. It is also known as citral, geranial, neral, geranialdehyde.

Citral is present in the oils of several plants, including lemon myrtle, lemongrass, verbena, lime, lemon and orange. Geranial has a pronounced lemon odor. Neral's lemon odor is not as intense, but sweet. Citral is primarily used in perfumery for its citrus quality. Citral is also used as a flavor and for fortifying lemon oil. It has strong antimicrobial qualities, and pheromonal effects in insects. Citral is used in the synthesis of vitamin A, ionone and methylionone.

In another embodiment, the terpene/terpenoid includes humulene. Humulene, also known as α-humulene or α-caryophyllene, is a naturally occurring monocyclic sesquiterpene (C₁₅H₂₄), which is an 11-membered ring consisting of 3 isoprene units containing three nonconjugated C═C double bonds, two of them being triply substituted and one being doubly substituted. It was first found in the essential oils of Humulus lupulus (hops). Humulene is an isomer of β-caryophyllene, and the two are often found together as a mixture in many aromatic plants.

Humulene has been shown to produce anti-inflammatory effects in mammals, which demonstrates potential for management of inflammatory diseases. It produces similar effects to dexamethasone, and was found to decrease the edema formation caused by histamine injections. Humulene produced inhibitory effects on tumor necrosis factor-α (TNFα) and interleukin-1.beta. (IL1B) generation in carrageenan-injected rats. In Chinese medicine, it is blended with β-caryophyllene and used as a remedy for inflammation.

Other exemplary terpenes and terpenoids include menthol, eucalyptol, borneol, pulegone, sabinene, terpineol, and thymol. In one embodiment, an exemplary terpene/terpenoid is eucalyptol.

C. NSAIDs

NSAIDs are the second-largest category of pain management treatment options in the world. The global pain management market was estimated at $22 billion in 2011, with $5.4 billion of this market being served by NSAID's. The U.S. makes up over one-half of the global market. The opioids market (such as morphine) form the largest single pain management sector but are known to be associated with serious dependence and tolerance issues.

Although NSAIDs are generally a safe and effective treatment method for pain, they have been associated with a number of gastrointestinal problems including dyspepsia and gastric bleeding.

Delivery of NSAIDs through the compositions and methods of the present invention will provide the beneficial properties of pain relief with lessened negative gastrointestinal effects, and also deliver lower dosages of active ingredients in order to provide pain management outcomes across a variety of indications.

Accordingly, in other aspects, within the compositions and methods of the present invention, the lipophilic active agent is an NSAID, particularly wherein the NSAID is selected from the group consisting of acetylsalicylic acid, ibuprophen, acetaminophen, diclofenac, indomethacin, and piroxicam.

In some aspects, the NSAID is a COX inhibitor, e.g., a selective COX inhibitor, e.g., a COX-2 inhibitor, e.g., celecoxib, deracoxib, valdecoxib, rofecoxib, tilmacoxib, or other similar known compounds, especially celecoxib, including its various known crystalline forms and various salts thereof (e.g., crystalline forms I, II, III, IV and N). In some aspects, active agents within the compositions according to the present invention are selective COX-2 inhibitors, which are known to be useful for treating: inflammation, colorectal polyps (because they have effects on abnormally dividing cells such as those of precancerous colorectal polyps), menstrual cramps, sports injuries, osteoarthritis, rheumatoid arthritis, and pain, e.g., acute pain, and for reducing the risk of peptic ulceration. Aspects of the invention are suitable for use with crystalline or amorphous forms of active ingredients.

In one aspect, the active agent is celecoxib, which is a selective COX-2 inhibitor having about 7.6-times higher affinity towards COX-2 than towards COX-1. Thus the anti-inflammatory activity of celecoxib is only rarely accompanied with gastrointestinal side effects which are often experienced with non-selective non-steroidal anti-inflammatory active ingredients.

D. Vitamins

The global vitamin and supplement market is worth $68 billion according to Euromonitor. The category is both broad and deep, comprised of many popular and some lesser known substances. Vitamins in general are thought to be an $8.5 billion annual market in the U.S. The U.S. is the largest single national market in the world, and China and Japan are the 2^{nd} and 3^{rd} largest vitamin markets.

The four most common fat-soluble vitamins are: vitamin A (retinol), vitamin D (calciferol), vitamin E (tocopherol), and vitamin K (phylloquinone and menaquinone).

Vitamin E is fat soluble and can be incorporated into cell membranes which can protect them from oxidative damage. Global consumption of natural source vitamin E was 10,900 metric tons in 2013 worth $611.9 million.

Accordingly, in other aspects, within the compositions and methods of the present invention, the lipophilic active agent is a fat soluble vitamin, particularly wherein the fat soluble vitamin is vitamin A, D, E, or K.

E. Nicotine Compounds

Nicotine is a natural ingredient in tobacco leaves where it acts as a botanical insecticide (Hukkanen et al. (2005) Pharmacological Reviews 57:79-115). Comprising about 95% of the total alkaloid content of commercial cigarette tobacco, nicotine comprises about 1.5% by weight of commercial cigarette tobacco (Hukkanen et al. (2005) Pharmacological Reviews 57:79-115). Although oral snuff and pipe tobacco contain concentrations of nicotine similar to cigarette tobacco, cigar and chewing tobacco typically contain only about half of the nicotine concentration of cigarette tobacco (Hukkanen et al. (2005) Pharmacological Reviews 57:79-115). An average tobacco rod typically contains 10 to 14 mg of nicotine (Hukkanen et al. (2005) Pharmacological Reviews 57:79-115), and on average about 1 to 1.5 mg of nicotine is absorbed systemically during smoking (Hukkanen et al. (2005) Pharmacological Reviews 57:79-115). The nicotine in tobacco is largely the levorotary (S)-isomer, only 0.1 to 0.6% of total nicotine content is (R)-nicotine (Hukkanen et al. (2005) Pharmacological Reviews 57:79-115). The (R)-nicotine content of tobacco smoke is higher, with up to 10% of nicotine in smoke reported to be (R)-isomer, and thought to be attributed to racemization occurring during combustion (Hukkanen et al. (2005) Pharmacological Reviews 57:79-115).

More than 99% of all nicotine that is consumed worldwide is delivered through smoking cigarettes. Approximately 6,000,000 deaths per year, worldwide, are attributed primarily to the delivery of nicotine through the act of smoking according to the Centers for Disease Control and Prevention, which also estimates that over $170 billion per year is spent just in the U.S. on direct medical care costs for adult smokers. In any twelve month period, 69% of U.S. adult smokers want to quit smoking and 43% of U.S. adult smokers have attempted to quit.

Worldwide, retail cigarette sales were worth $722 billion in 2013, with over 5.7 trillion cigarettes sold to more than 1 billion smokers. It would be desirable in the art to provide further methods for altering the character and nature of tobacco (and tobacco compositions and formulations) useful in smoking articles and/or or smokeless tobacco products, including enhancement of bioavailability of active agents, masking of unpleasant tastes, and the incorporation of additional active agents. Furthermore, the delivery of nicotine to satisfy current demand via the compositions and methods of the present invention, can in part alleviate the consumer demand for cigarettes. Since most of the adverse health outcomes of nicotine consumption are associated with the delivery method and only to a lesser degree to the actual ingestion of nicotine, a vast positive community health outcome can be achieved through the reduction in smoking cigarettes.

Accordingly, in other aspects, within the compositions and methods of the present invention, the lipophilic active agent is a nicotine compound.

As used herein, “nicotine compound” or “source of nicotine” often refers to naturally-occurring or synthetic nicotine compound unbound from a plant material, meaning the compound is at least partially purified and not contained within a plant structure, such as a tobacco leaf. Most preferably, nicotine is naturally-occurring and obtained as an extract from a Nicotiana species (e.g., tobacco). The nicotine can have the enantiomeric form S(−)-nicotine, R(+)-nicotine, or a mixture of S(−)-nicotine and R(+)-nicotine. Most preferably, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis. Despite the fact that nicotine can be extracted from Nicotiana species, it is highly preferred that the nicotine (and the composition and products produced in accordance with the present invention) are virtually or essentially absent of other components obtained from or derived from tobacco.

Nicotine compounds can include nicotine in free base form, salt form, as a complex, or as a solvate. See, for example, the discussion of nicotine in free base form in US Pat. Pub. No. 2004/0191322 to Hansson, which is incorporated herein by reference. At least a portion of the nicotine compound can be employed in the form of a resin complex of nicotine, where nicotine is bound in an ion exchange resin, such as nicotine polacrilex. See, for example, U.S. Pat. No. 3,901,248 to Lichtneckert et al., which is incorporated herein by reference. At least a portion of the nicotine can be employed in the form of a salt. Salts of nicotine can be provided using the types of ingredients and techniques set forth in U.S. Pat. No. 2,033,909 to Cox et al. and U.S. Pat. No. 4,830,028 to Lawson et al., and Perfetti, Beitrage Tabakforschung Int., 12: 43-54 (1983), which are incorporated herein by reference. See, also, U.S. patent application Ser. No. 12/769,335 to Brinkley et al., filed Apr. 28, 2010, which is incorporated herein by reference. Additionally, salts of nicotine have been available from sources such as Pfaltz and Bauer, Inc. and K&K Laboratories, Division of ICN Biochemicals, Inc.

Exemplary pharmaceutically acceptable nicotine salts include nicotine salts of tartrate (e.g., nicotine tartrate and nicotine bitartrate) chloride (e.g., nicotine hydrochloride and nicotine dihydrochloride), sulfate, perchlorate, ascorbate, fumarate, citrate, malate, lactate, aspartate, salicylate, tosylate, succinate, pyruvate, and the like; nicotine salt hydrates (e.g., nicotine zinc chloride monohydrate), and the like. Additional organic acids that can form salts with nicotine include formic, acetic, propionic, isobutyric, butyric, alpha-methylbutyric, isovaleric, beta-methylvaleric, caproic, 2-furoic, phenylacetic, heptanoic, octanoic, nonanoic, oxalic, malonic, and glycolic acid, as well as other fatty acids having carbon chains of up to about 20 carbon atoms.

In many embodiments, the nicotine compound will be present in multiple forms. For example, the nicotine can be employed within the composition as a mixture of at least two salts (e.g., two different organic acid salts, such as a mixture of nicotine bitartrate and nicotine levulinate), as at least two salts that are segregated within the composition, in a free base form and salt form, in a free base form and a salt form that are segregated within the composition, in a salt form and in a complexed form (e.g., a resin complex such as nicotine polacrilex), in a salt for and in a complexed form that are segregated with in the composition, in a free base form and a complexed form, in a free base form and a complexed form that are segregated within the composition, or the like. As such, each single dosage unit or piece (e.g., gum piece, lozenge, sachet, film strip, etc.) can incorporate at least two forms of nicotine.

A nicotine compound, in particular a compound such as nicotine, also can be employed in combination with other so-called tobacco alkaloids (i.e., alkaloids that have been identified as naturally occurring in tobacco). For example, nicotine, as employed in accordance with the present invention, can be employed in combination with nornicotine, anatabine, anabasine, and the like, and combinations thereof. See, for example, Jacob et al., Am. J. Pub. Health, 5: 731-736 (1999), which is incorporated herein by reference.

The compositions of the invention most preferably possess a form that is pharmaceutically effective and pharmaceutically acceptable. That is, the composition most preferably does not incorporate to any appreciable degree, or does not purposefully incorporate, significant amounts of components of tobacco, other than nicotine. As such, pharmaceutically effective and pharmaceutically acceptable compositions do not include tobacco in parts or pieces, processed tobacco components, or many of the components of tobacco traditionally present within tobacco-containing cigarettes, cigars, pipes, or smokeless forms of tobacco products. Highly preferred compositions that are derived by extracting naturally-occurring nicotine from tobacco include less than 5 weight percent of tobacco components other than nicotine, more often less than about 0.5 weight percent, frequently less than about 0.25 weight percent, and typically are entirely absent or devoid of components of tobacco, processed tobacco components, or components derived from tobacco, other than nicotine, based on the total weight of the composition.

In some embodiments, the nicotine compound is selected from the group consisting of nicotine and a nicotine derivative, wherein the nicotine derivative comprises a nicotine salt, a nicotine complex, a nicotine polacrilex, or combinations thereof.

Tobacco alkaloids include nicotine and nicotine-like or related pharmacologically active compounds such as nor-nicotine, lobeline and the like, as well as the free base substance nicotine and all pharmacologically acceptable salts of nicotine, including acid addition salts. “Nicotine compounds” as that term is used herein therefore includes all the foregoing tobacco alkaloids, as well as nicotine salts including but not limited to nicotine hydrogen tartrate and nicotine bitartrate dihydrate, as well as nicotine hydrochloride, nicotine dihydrochloride, nicotine sulfate, nicotine citrate, nicotine zinc chloride monohydrate, nicotine salicylate, nicotine oil, nicotine complexed with cyclodextrin, polymer resins such as nicotine polacrilex, nicotine resinate, and other nicotine-ion exchange resins, either alone or in combination.

The nicotine compounds also include nicotine analogs that include, but are not limited to the structures shown below for (s)-Nicotine, Nornicotine, (S)-Cotinine, B-Nicotyrine, (S)-Nicotene-N′-Oxide, Anabasine, Anatabine, Myosmine, B-Nornicotyrine, 4-(Methylamino)-1-(3-pyridyl)-1-butene (Metanicotine) cis or trans, N′-Methylanabasine, N′Methylanatabine, N′Methylmyosmine, 4-(Methylamino)-1-(3-pyridyl)-1-butanone (Pseudoxynicotine), and 2,3′-Bipyridyl (Hukkanen et al. (2005) Pharmacological Reviews 57:79-115):

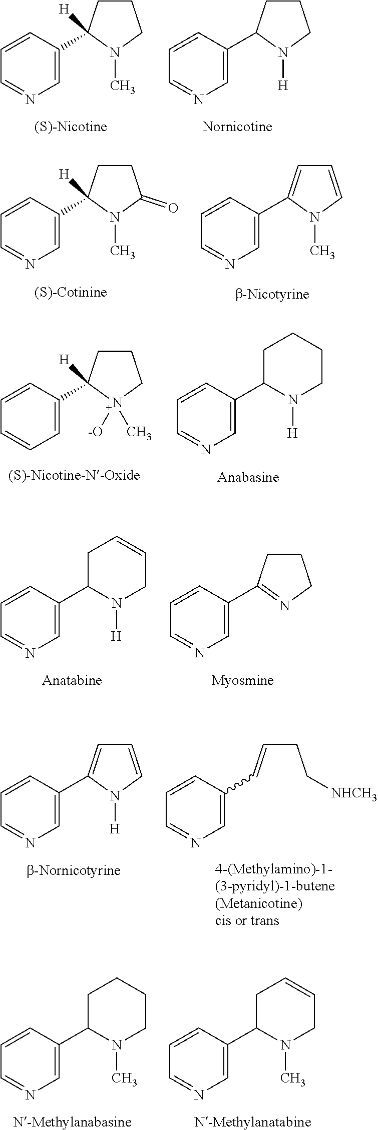

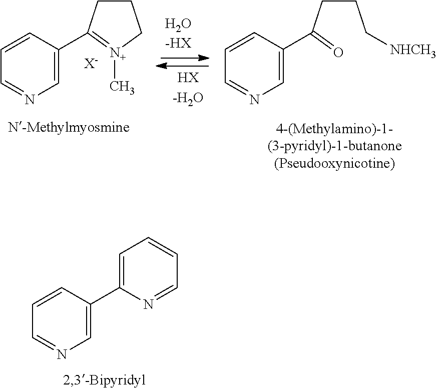

Nicotine compounds also include nicotine bitartrate, cytisine, nicotine polacrilex, nornicotine, nicotine 1-N-oxide, metanicotine, nicotine imine, nicotine N-glucuronide, N-methylnicotinium, N-n-decylnicotinium, 5′-cyanonicotine, 3,4-dihydrometanicotine, N′-methylnicotinium, N-octanoylnornicotine, 2,3,3a,4,5,9b-hexahydro-1-methyl-1H-pyrrolo(3,2-h)isoquinoline, 5-isothiocyanonicotine, 5-iodonicotine, 5′-hydroxycotinine-N-oxide, homoazanicotine, nicotine monomethiodide, N-4-azido-2-nitrophenylnornicotine, N-methylnornicotinium, nicotinium molybdophosphate resin, N-methyl-N′-oxonicotinium, N′-propylnornicotine, pseudooxynicotine, 4′-methylnicotine, 5-fluoronicotine, K(s-nic)5(Ga2(N,N′-bis-(2,3-dihydroxybenzoyl)-1,4-phenylenediamine)3), 5-methoxynicotine, 1-benzyl-4-phenylnicotinamidinium, 6-n-propylnicotine, SIB1663, 6-hydroxynicotine, N-methyl-nicotine, 6-(2-phenylethyl)nicotine, N′-formylnornicotine, N-n-octylnicotinium, N-(n-oct-3-enyl)nicotinium, N-(n-dec-9-enyl)nicotinium, 5′-acetoxy-N′-nitrosonornicotine, 4-hydroxynicotine, 4-(dimethylphenyl silyl)nicotine, N′-carbomethoxynornicotine, and N-methylnicoton.

The nicotine compound may be used in one or more distinct physical forms well known in the art, including free base forms, encapsulated forms, ionized forms and spray-dried forms.

Additional description regarding the chemistry, absorption, metabolism, kinetics and biomarkers of nicotine is described in Hukkanen et al. (2005) Pharmacological Reviews 57:79-115 and Benowitz et al. (2009) Handb. Exp. Pharmacol. 192:29-60, which are both incorporated herein in their entireties.

The compositions also include nicotine compounds characterized as selective agonists to nicotinic receptor subtypes that are present in the brain, or that can otherwise be characterized as a compound that modulates nicotinic receptor subtypes of the CNS. Various nicotinic receptor subtypes are described in Dwoskin et al., Exp. Opin. Ther. Patents, 10: 1561-1581 (2000); Huang et al., J. Am. Chem. Soc., 127: 14401-14414 (2006); and Millar, Biochem. Pharmacol., 78: 766-776 (2009); which are incorporated herein by reference. Representative compounds that can be characterized as other nicotine compounds for purposes of this invention are set forth in Schmitt et al., Annual Reports in Med. Chem. 35: 41-51 (2000); and Arneric et al., Biochem. Pharmacol., 74: 1092-1101 (2007); which are incorporated herein by reference.

In one aspect, the nicotine compound can be a compound has selectivity to the α₇ (alpha 7) nicotinic receptor subtype, and preferably is an agonist of the α₇ nicotinic receptor subtype. Several compounds having such α₇ receptor subtype selectivity have been reported in the literature. For example, various compounds purported to have selectivity to the α₇ nicotinic receptor subtype are set forth in Malysz et al., Assay Drug Dev. Tech., August: 374-390 (2009). An example of one such nicotine compound is N-[(2S,3S)-2-(pyridin-3-ylmethyl)-1-azabicyclo[2.2.2]oct-3-yl]-1-benzofur-an-2-carboxamide (also known as TC-5619). See, for example, Hauser et al., Biochem. Pharmacol., 78: 803-812 (2009). Another representative is compound is (5aS,8S,10aR)-5a,6,9,10-Tetrahydro,7H,11H-8,10a-methanopyrido [2′,3′:5,6]pyrano[2,3-d]azepine (also known as dianicline or SSR591813 or SSR-591,813). See, for example, Hajos et al., J. Pharmacol. Exp. Ther., 312: 1213-1222 (2005). Another representative compound is 1,4-Diazabicyclo[3.2.2]nonane-4-carboxylic acid, 4-bromophenyl ester (also known as SSR180711). See, for example, Biton et al., Neuropsychopharmacol., 32: 1-16 (2007). Another representative compound is 3-[(3E)-3-[(2,4-dimethoxyphenyl)methylidene]-5,6-dihydro-4H-pyridin-2-yl]pyridine (also known as GTS-21). See, for example, U.S. Pat. No. 5,516,802 to Zoltewicz et al. and U.S. Pat. No. 5,741,802 to Kem et al. Another representative compound is 2-methyl-5-(6-phenyl-pyridazin-3-yl)-octahydro-pyrrolo[3,4-c]pyrrole (also known as A-582941). See, for example, Thomsen et al., Neuroscience, 154: 741-753 (2008). Another representative compound is (5S)-spiro[1,3-oxazolidine-5,8′-1-azabicyclo[2.2.2]octane]-2-one (also known as AR-R-17779 or AR-R-17779). See, for example, Li et al., Neuropsycopharmacol., 33: 2820-2830 (2008). Another representative compound is N-[(3R)-1-azabicyclo[2.2.2]oct-3-yl]-4-chlorobenzamide (also known as PNU-282,987). See, for example, Siok et al., Eur. J. Neurosci., 23: 570-574 (2006). Another representative compound is 5-morpholin-4-yl-pentanoic acid (4-pyridin-3-yl-phenyl)-amide (also known as WAY-317,538 or SEN-12333). See, for example, Roncarati et al., J Pharmacol. Exp. Ther., 329: 459-468 (2009). Yet other examples are compounds are those designated as EVP-6124 and EVP-4473 by Envivo Pharmaceuticals, Inc., TC-6987 by Targacept, Inc. and MEM3454 by Memory Pharmaceuticals Corp. The foregoing cited references are incorporated herein by reference.

In one aspect, the nicotine compound can be a compound that has selectivity to the α₄β₂ (alpha 4 beta 2) nicotinic receptor subtype, and preferably is an agonist of the α₄β₂ nicotinic receptor subtype. Several compounds having such α₄β₂ receptor subtype selectivity have been reported in the literature. An example of one such nicotine compound is known as 7,8,9,10-tetrahydro-6,10-methano-6H-pyrazino(2,3-h)(3) benzazepine (also known as varenicline and in the form of varenicline tartrate which is the active ingredient of a product commercially marketed under the tradename Chantix or Champix by Pfizer). See, for example, Jorenby et al., JAMA, 296: 56-63 (2006) and US Pat. Pub. No. 2010/0004451 to Ahmed et al. Another representative compound is (2S,4E)-5-(5-isopropoxypyridin-3-yl)-N-methylpent-4-en-2-amine (also known as ispronicline or AZD-3480 of AstraZeneca or TC-1734 of Targacept, Inc. (Winston-Salem, N.C., USA)). See, for example, Dunbar et al., Psychopharmacol. (Berlin), 191: 919-929 (2007). Another representative compound is [3-(2(S))-azetidinylmethoxy)pyridine] dihydrochloride, (also known as A-85380). See, for example, Schreiber, Psychopharmacol., 159:248-257 (2002). Another representative compound is (5 aS,8S,10aR)-5a,6,9,10-Tetrahydro,7H,11H-8,10a-methanopyrido [2′,3′:5,6]pyrano[2,3-d]azepine (also known as SSR591813). See, for example, Cohen et al., Neuroscience, Pres. No. 811.5 (2002); and Cohen et al., J. Pharmacol. Exp. Ther., 306: 407-420 (2003). Another representative compound is known as A-969933. See, for example, Zhu et al., Biochem. Pharmacol., 78: 920 (2009). Other representative compounds are known as S35836-1 and S35678-1. See, for example, Lockhart et al., Neuroscience, Pres. No. 684.9 (2002). Yet other examples are compounds are those designated as 3-(5,6-Dichloro-pyridin-3-yl)-1S,5S-3,6-diazabicyclo[3.2.0]heptane (also known as Sofinicline or ABT-894) by Abbott Laboratories; AZD1446 by AstraZeneca and TC-6499 by Targacept, Inc. The foregoing cited references are incorporated herein by reference.

In some cases, the nicotine can be liquid nicotine. Liquid nicotine can be purchased from commercial sources, whether tobacco-derived or synthetic. Tobacco-derived nicotine can include one or more other tobacco organoleptic components other than nicotine. The tobacco-derived nicotine can be extracted from raw (e.g., green leaf) tobacco and/or processed tobacco. Processed tobaccos can include fermented and unfermented tobaccos, dark air-cured, dark fire cured, burley, flue cured, and cigar filler or wrapper, as well as the products from the whole leaf stemming operation. The tobacco can also be conditioned by heating, sweating and/or pasteurizing steps as described in U.S. Publication Nos. 2004/0118422 or 2005/0178398. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. By processing the tobacco prior to extracting nicotine and other organoleptic components, the tobacco-derived nicotine may include ingredients that provide a favorable experience. The tobacco-derived nicotine can be obtained by mixing cured tobacco or cured and fermented tobacco with water or another solvent (e.g., ethanol) followed by removing the insoluble tobacco material. The tobacco extract may be further concentrated or purified. In some cases, select tobacco constituents can be removed. Nicotine can also be extracted from tobacco in the methods described in the following patents: U.S. Pat. Nos. 2,162,738; 3,139,436; 3,396,735; 4,153,063; 4,448,208; and 5,487,792.

Liquid nicotine can be pure, substantially pure, or diluted prior to mixing it with soluble fiber. Soluble fiber dissolves in water at ambient temperature. Insoluble fiber does not dissolve in water at ambient temperature. Soluble fibers can attract water and form a gel. Not only are many soluble fibers safe for consumption, but some soluble fibers are used as a dietary supplement. As a dietary supplement, soluble fiber can slow down digestion and delay the emptying of a stomach. Instead of using soluble fiber as a mere additive, however, nicotine lozenges provided herein include a matrix of soluble fiber, which can dissolve to provide access to nicotine (and optionally other additives) included in the soluble-fiber matrix.

For liquid nicotine, a diluting step is optional. In some cases, liquid nicotine is diluted to a concentration of between 1 weight percent and 75 weight percent prior to mixing the liquid nicotine with soluble fiber. In some cases, liquid nicotine is diluted to a concentration of between 2 weight percent and 50 weight percent prior to mixing the liquid nicotine with soluble fiber. In some cases, liquid nicotine is diluted to a concentration of between 5 weight percent and 25 weight percent prior to mixing the liquid nicotine with soluble fiber. For example, liquid nicotine can be diluted to a concentration of about 10 weight percent prior to mixing the liquid nicotine with soluble fiber.

F. Phosphodiesterase Type 5 Inhibitors

Phosphodiesterase type 5 inhibitors (PDE5 inhibitors) block the degradative action of cGMP-specific phosphodiesterase type 5 (PDE5) on cyclic GMP in the smooth muscle cells lining the blood vessels supplying the corpus cavernosum of the penis. These drugs, including vardenafil (Levitra®), sildenafil (Viagra®), and tadalafil (Cialis®), are administered orally for the treatment of erectile dysfunction and were the first effective oral treatment available for the condition.

PDE5 inhibitors have also been studied for other clinical use as well, including cardiovascular and heart diseases. For example, because PDE5 is also present in the arterial wall smooth muscle within the lungs, PDE5 inhibitors have also been explored for lung diseases such as pulmonary hypertension and cystic fibrosis. Pulmonary arterial hypertension, a disease characterized by sustained elevations of pulmonary artery pressure, which leads to an increased incidence of failure of the right ventricle of the heart, which in turn can result in the blood vessels in the lungs become overloaded with fluid. Two oral PDE5 inhibitors, sildenafil (Revatio®) and tadalafil (Adcirca®), are approved for the treatment of pulmonary arterial hypertension. PDE5 inhibitors have been found to have activity as both a corrector and potentiator of CFTR protein abnormalities in animal models of cystic fibrosis disease (Lubamba et al., Am. J. Respir. Crit. Care Med. (2008) 177:506-515, Lubamba et al., J. Cystic Fibrosis (2012) 11:266-273). Sildenafil has also been studied as a potential anti-inflammatory treatment for cystic fibrosis. Oral PDE5 inhibitors have also been reported to have anti-remodeling properties and to improve cardiac inotropism, independent of afterload changes, with a good safety profile (Giannetta et al., BMC Medicine (2014) 12:185). However, oral administration of PDE5 inhibitors results in poor and variable bioavailability and also extensive metabolism in the liver (Sandqvist et al., Eur. J. Clin. Pharmacol. (2013) 69:197-207; Mehrotra, Intl. J. Impotence Res. (2007) 19:253-264). If oral doses are increased beyond certain levels, the incidence of systemic side effects increase which prevents the acceptable use of these drugs. (Levitra EMEA Scientific Discussion Document, 2005).

Accordingly, in other aspects, within the compositions and methods of the present invention, the PDE5 inhibitor may include, but is not limited to, avanafil, lodenafil, mirodenafil, sildenafil (or analogs thereof, for example, actetildenafil, hydroxyacetildenafil, or dimethyl-sildenafil), tadalafil, vardenafil, udenafil, acetildenafil, or thiome-thisosildenafil. The structures of these compounds are respectively shown below:

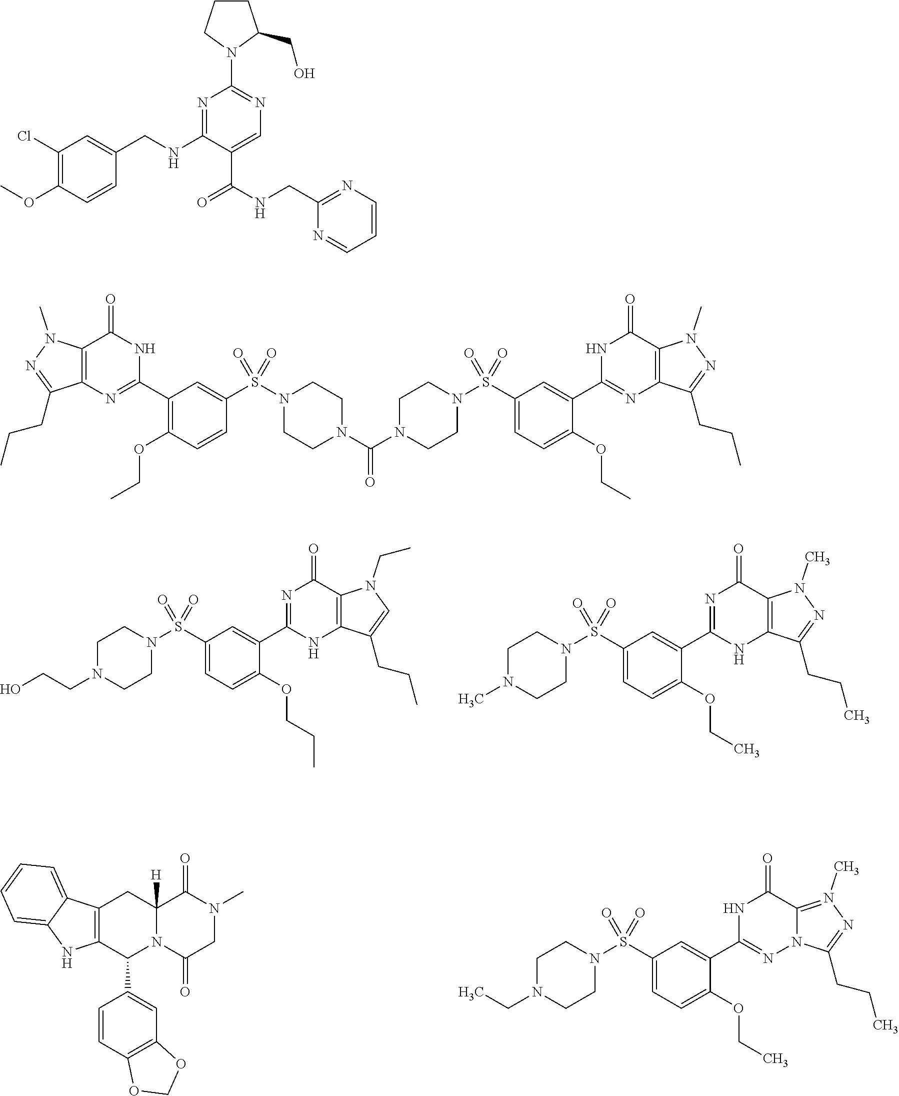

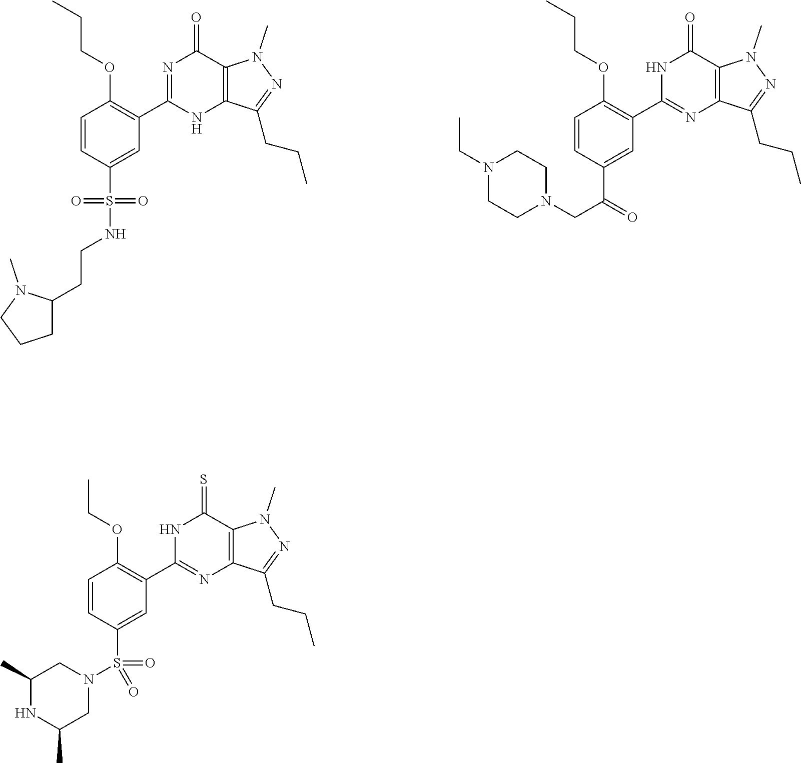 [SMILES: CCCOc1ccc(S(=O)(=O)NCCC2CCCN2C)cc1-c1nc(=O)c2c([nH]1)c(CCC)nn2C ; CCCOc1ccc(C(=O)CN2CCN(CC)CC2)cc1-c1nc2c(CCC)nn(C)c2c(=O)[nH]1 ; CCCc1nn(C)c2c(=S)[nH]c(-c3cc(S(=O)(=O)N4C[C@@H](C)N[C@@H](C)C4)ccc3OCC)nc12]

G. Maca Extract

Lepidium meyenii (Maca, maca-maca, maino, ayak chichira, and ayak willku) is a Peruvian plant of the Brassicaceae family cultivated for more than 2000 years. Its main active principles are alkaloids (Macaridine, Lepidiline A and B); bencil-isotiocyanate and glucosinolates; macamides, beta-ecdysone and fitosterols. These substances activate ATP synthesis which confers energizing properties. They also diminish variations in homeostasis produced by stress because they reduce corticosterone's high levels; prevent glucose diminution and the increase of suprarenal glands' weight due to stress. They also restore homeostasis and improve energy (Lopez-Fando et al. (2004) Phytother Res. 18:471-4). A double blind placebo-controlled, randomized, parallel trial study in which active treatment with different doses of Lepidium meyenii was compared with placebo showed an improvement in sexual desire. (Gonzales et al. (2002) Andrologia 34:367-72). Lepidium meyenii also improves sperm production and sperm motility by mechanisms not related to LH, FSH, PRL, T and E2 (Gonzales et al. (2001) Asian J. Androl. 3:301-3).

H. Steroid Hormones

In some embodiments, the active agent is a steroid, including hormones and sex hormones. The term “sex hormone” refers to natural or synthetic steroid hormones that interact with vertebrate androgen or estrogen receptors, such as estrogens, anti-oestrogens (or SERMs), androgens, anti-androgens, progestins, and mixtures thereof.

For example, steroid hormones suitable for use in the compositions described herein include the numerous natural and synthetic steroid hormones, including androgens, estrogens, and progestagens and derivatives thereof, such as dehydroepiandrosterone (DHEA), androstenedione, androstenediol, dihydrotestosterone, testosterone, progesterone, progestins, oestriol, oestradiol. Other suitable steroid hormones include glucocorticoids, thyroid hormone, calciferol, pregnenolone, aldosterone, cortisol, and derivatives thereof. Suitable steroid hormones especially include the sexual hormones having estrogenic, progestational, androgenic, or anabolic effects, such as estrogen, estradiol and their esters, e.g., the valerate, benzoate, or undecylate, ethinylestradiol, etc.; progestogens, such as norethisterone acetate, levonorgestrel, chlormadinone acetate, cyproterone acetate, desogestrel, or gestodene, etc.; androgens, such as testosterone and its esters (propionate, undecylate, etc.), etc.; anabolics, such as methandrostenolone, nandrolone and its esters.

i. Estrogens

Estrogens refer to a group of endogenous and synthetic hormones that are important for and used for tissue and bone maintenance. Estrogens are endocrine regulators in the cellular processes involved in the development and maintenance of the reproductive system. The role of estrogens in reproductive biology, the prevention of postmenopausal hot flashes, and the prevention of postmenopausal osteoporosis are well established. Estradiol is the principal endogenous human estrogen, and is found in both women and men.

The biological actions of estrogens and antiestrogens are manifest through two distinct intracellular receptors, estrogen receptor alpha (ERα) and estrogen receptor beta (ERβ). Endogenous estrogens are typically potent activators of both receptor subtypes. For example estradiol acts as an ERα agonist in many tissues, including breast, bone, cardiovascular and central nervous system tissues. Selective estrogen receptor modulators commonly act differently in different tissues. For example, a SERM may be an ERα antagonist in the breast, but may be a partial ERα agonist in the uterus, bone and cardiovascular systems. Compounds that act as estrogen receptor ligands are, therefore, useful in treating a variety of conditions and disorders.

As used herein, “estrogen” includes estrogenic steroids such as estradiol (17-β-estradiol), estradiol benzoate, estradiol 17 β-cypionate, estropipate, equilenin, equilin, estriol, estrone, ethinyl estradiol, conjugated estrogens, esterified estrogens, phytoestrogens, semi-natural estrogens such as estradiol valerate, synthetic estrogens such as ethinyl-estradiol, and mixtures thereof.

In some embodiments, a pharmaceutical composition is provided for topical administration to a skin surface comprising water, and at least one therapeutically active agent selected from the estrogens. In some embodiments the compositions and methods of the invention further comprise an alcohol and a fatty acid ester. In some embodiments, a pharmaceutical composition is provided for topical administration to a skin surface comprising water and at least one therapeutically active agent being estradiol. In some embodiments, the compositions and methods of the invention further comprise an alcohol and a fatty acid ester. In particular embodiments of such compositions when the active agent is estradiol, the compositions and methods do not further comprise the combination of progesterone, propylene glycol, oleic acid, ethyl oleate, ethanol, hydroxypropylcellulose and purified water.

ii. Anti-Estrogens

Anti-estrogens are a class of pharmaceutically active agents now referred to as Selective Estrogen Receptors Modulators (SERMs), which were generally understood to be compounds capable of blocking the effect of estradiol without displaying any estrogenic activity of their own. Such a description is now known to be incomplete, however. The term SERM has been coined to describe compounds that, in contrast to pure estrogen agonists or antagonists, have a mixed and selective pattern of estrogen agonist-antagonist activity, which largely depends on the targeted tissue. The pharmacological goal of these drugs is to produce estrogenic actions in those tissues where these actions are beneficial (such as bone, brain, liver) and to have either no activity or antagonistic activity in tissues such as breast and endometrium, where estrogenic actions (cellular proliferation) might be deleterious.

In specific embodiments, the anti-estrogens (SERMs) are selected from the group consisting of endoxifen, droloxifene, clomifene, raloxifene, tamoxifen, 4-OH tamoxifen, toremifene, danazol, and pharmaceutically acceptable salts thereof. In a more particular embodiment, a pharmaceutical composition is provided for topical administration to a skin surface comprising water, at least one therapeutically active agent selected from the anti-oestrogens (SERMs) selected from the group consisting of clomifene, raloxifene, droloxifene, endoxifen or the pharmaceutically acceptable salts thereof, an alcohol, and a fatty acid ester.

In a particular embodiment, a pharmaceutical composition is provided for topical administration to a skin surface comprising water, at least one therapeutically active agent selected from the anti-estrogens (SERMs). In some aspects the composition further comprises an alcohol and a fatty acid ester.

iii. Androgens

Testosterone is the main androgenic hormone formed in the testes. Testosterone therapy is currently indicated for the treatment of male hypogonadism. It is also under investigation for the treatment of wasting conditions associated with AIDS and cancer, testosterone replacement in men over the age of 60, osteoporosis, combination hormone replacement therapy for women and male fertility control.

Orally administered testosterone is largely degraded in the liver, and is therefore not a viable option for hormone replacement since it does not allow testosterone to reach systemic circulation. Further, analogues of testosterone modified to reduce degradation (e.g., methyltestosterone and methandrostenolone) have been associated with abnormalities in liver function, such as elevation of liver enzymes and conjugated bilirubin. Injected testosterone produces wide peak-to-trough variations in testosterone concentrations that do not mimic the normal fluctuations of testosterone, and makes maintenance of physiological levels in the plasma difficult. Testosterone injections are also associated with mood swings and increased serum lipid levels. Injections require large needles for intramuscular delivery, which leads to diminished patient compliance due to discomfort.

To overcome these problems, transdermal delivery approaches have been developed to achieve therapeutic effects in a more patient friendly manner. For example, U.S. Pat. No. 5,460,820 discloses a testosterone-delivering patch for delivering 50 to 500 μg/day of testosterone to a woman. In addition, U.S. Pat. No. 5,152,997 discloses a device comprising a reservoir of testosterone with a skin permeation enhancer and a means for maintaining the reservoir in diffusional communication with the skin, such as an adhesive carrier device or a basal adhesive layer.

In some embodiments, androgens may be selected from the group consisting of the natural androgen, testosterone, and its semi-natural or synthetic derivatives, for instance methyltestosterone; physiological precursors of testosterone such as dehydroepiandrosterone or DHEA, or alternatively prasterone and its derivatives, for instance DHEA sulphate, Δ-4-androstenedione and its derivatives; testosterone metabolites, for instance dihydrotestosterone (DHT) obtained after the enzymatic action of 5-α-reductases; or substances with an androgenic-type effect, such as tibolone. In some aspects the composition further comprises an alcohol and a fatty acid ester.

iv. Anti-Androgens

In some embodiments, anti-androgens are selected from the group consisting of steroidal compounds such as cyproterone acetate and medroxyprogesterone, or non-steroidal compounds such as flutamide, nilutamide or bicalutamide. In some aspects the composition further comprises an alcohol and a fatty acid ester.

v. Progestins and Progesterone

The term “progesterone” as used herein refers to a member of the progestin family and comprises a 21 carbon steroid hormone. Progesterone is also known as D4-pregnene-3,20-dione; 4-pregnene-3,20-dione; or pregn-4-ene-3,20-dione. A progestin is a molecule whose structure is related to that of progesterone, is synthetically derived, and retains the biologically activity of progesterone. Representative synthetic progestin include, but are not limited to, modifications that produce 17a-OH esters (i.e., 17 a-hydroxyprogesterone caproate), as well as, modifications that introduce 6 a-methyl, 6-Me, 6-ene, and 6-chloro substituents onto progesterone (i.e., medroxyprogesterone acetate, megestrol acetate, and chlormadinone acetate).

In some embodiments, progestin(s) used in the compositions and methods described herein may be selected from the group consisting of natural progestins, progesterone or its derivatives of ester type, and synthetic progestins of type 1, 2 or 3. The first group comprises molecules similar to progesterone or the synthetic progestins 1 (SP1) (pregnanes), for example the progesterone isomer (retroprogesterone), medrogesterone, and norprogesterone derivatives (demegestone or promegestone). The second group comprises 17α-hydroxy-progesterone derivatives or synthetic progestins 2 (SP2) (pregnanes), for example cyproterone acetate and medroxyprogesterone acetate. The third group comprises norsteroids or synthetic progestins 3 (SP3), (estranes or nor-androstanes). These are 19-nortestosterone derivatives, for example norethindrone. This group also comprises molecules of gonane type, which are derived from these nor-androstanes or estranes and have a methyl group at C18 and an ethyl group at C13. Examples that may be mentioned include norgestimate, desogestrel (3-ketodesogestrel) or gestodene. Tibolone, which has both progestin and androgenic activity, may also advantageously be selected in the compositions and methods described herein. In some aspects the composition further comprises an alcohol and a fatty acid ester. In some embodiments of such compositions, when the active agent is progesterone, the composition does not further comprise the combination of estradiol, propylene glycol, oleic acid, ethyl oleate, ethanol, hydroxypropylcellulose and purified water. In some embodiments, the therapeutically active agent in the compositions and methods is a progestin, an estrogen or a combination of the two.

I. Fentanyl

Fentanyl (also known as fentanil) is a potent synthetic narcotic analgesic with a rapid onset and short duration of action. Fentanyl is a strong agonist at μ-opioid receptors. Fentanyl is manufactured under the trade names of SUBLIMAZE, ACTIQ, DUROGESIC, DURAGESIC, FENTORA, ONSOLIS INSTANYL, ABSTRAL, and others. Historically, fentanyl has been used to treat chronic breakthrough pain and is commonly used before procedures as an anesthetic in combination with a benzodiazepine. Fentanyl is approximately 100 times more potent than morphine with 100 micrograms of fentanyl approximately equivalent to 10 mg of morphine and 75 mg of pethidine (meperidine) in analgesic activity.

Suitable analogues of fentanyl include, without limitation, the following: alfentanil (trade name ALFENTA), an ultra-short-acting (five to ten minutes) analgesic; sufentanil (trade name SUFENTA), a potent analgesic for use in specific surgeries and surgery in heavily opioid-tolerant/opioid-dependent patients; remifentanil (trade name ULTIVA), currently the shortest-acting opioid, has the benefit of rapid offset, even after prolonged infusions; carfentanil (trade name WILDNIL) an analogue of fentanyl with an analgesic potency 10,000 times that of morphine and is used in veterinary practice to immobilize certain large animals such as elephants; and lofentanil an analogue of fentanyl with a potency slightly greater than carfentanil.

J. Buprenorphine

Buprenorphine (17-(cyclopropyl-methyl)-α-(1,1-dimethylethyl)-4,5-epoxy-18,19-dihydro-3-hydroxy-6-methoxy-α-methyl-6,14-ethenomorphinan-7-methanol) is an endoethylene morphinan derivative and a partial agonist of μ-opioid receptors with a strong analgesic effect. Buprenorphine is a partially synthetic opiate whose advantage over other compounds from this class of substance lies in a higher activity. This means that freedom from pain can be achieved in cancer or tumour patients with very unfavourable diagnosis, in the final stage, with daily doses of around 1 mg. A feature of buprenorphine in this context over the synthetic opioid fentanyl and its analogues is that the addictive potential of buprenorphine is lower than that of these compounds. A disadvantage is that, owing to the high molecular weight of buprenorphine, namely 467.64 daltons, it has been traditionally been difficult to effect its transdermal absorption.

K. Scopolamine

Scopolamine is a so-called antiemetic, it is preferably used to avoid nausea and vomiting, for example, arising from repeated passive changes in the balance occurring during traveling. Scopolamine is represented by the following chemical structure:

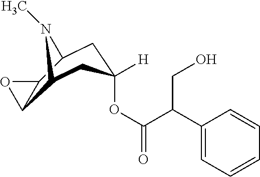

Scopolamine analogs are also encompassed by the compositions and methods of the present invention. It is understood that the phrase “scopolamine analogs” includes compounds that generally have the same backbone as scopolamine, but where various moieties have been substituted or replaced by other substituents or moieties. Some examples of scopolamine analogs that can be used in the compositions and methods disclosed herein include, but are not limited to, salts of scopolamine with various acids, such as hydrochloric acid, hydrobromic acid, hydroiodic acid, nitric acid, phosphoric acid, sulfuric acid, and the like. In one aspect, a suitable scopolamine analog can be scopolamine hydrobromide.

Additional examples of scopolamine analogs include, but are not limited to, N-alkylated analogs of scopolamine, that is, analogs containing an alkyl substituent attached to the nitrogen atom, forming a quaternary ammonium species. By “alkyl” is meant a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl, and the like. The alkyl group can also be substituted or unsubstituted.

Also included are other salts (e.g., pharmaceutically acceptable salts) of such N-alkylated scopolamine analogs.

Still further examples of scopolamine analogs include, but are not limited to, un-epoxylated analogs of scopolamine, that is, analogs where the epoxy group is removed. One example of such an analog is atropine. Like scopolamine, atropine has various salt and N-alkylated analogs. These atropine analogs are intended to be included by the phrase “scopolamine analogs.” As such, further examples of scopolamine analogs include, but are not limited to, analogs of atropine with various salts (e.g., atropine hydrobromide, atropine hydrochloride, and the like) and N-alkylated analogs of atropine (e.g., atropine methyl bromide). Also included are homatropine and its salts and N-alkylated analogs.

A list of suitable scopolamine analogs that can be used in the disclosed compositions and methods, including their commercial brand names, includes, but is not limited to, atropine, atropine hydrobromide, atropine oxide hydrochloride, atropine sulfate, belladonna, scopolamine, scopolamine hydrobromide, scopolamine methylbromide, scopolamine butylbromide, homatropine, ipratropium, tiotropium, hyoscyamine sulfate, methscopolamine, methscopolamine bromide, homatropine hydrobromide, homatropine methylbromide, hyoscyamine, hyoscyamine hydrobromide, hyoscyamine sulfate, propantheline bromide, anisotropine, anisotropine methylbromide, methantheline bromide, emepronium bromide, clindinium, clidinium bromide, hyoscine, hyoscine butylbromide, hyoscine hydrobromide, hyoscine methobromide, hyoscine methonitrite, hyoscyamine, hyoscyamine sulfate, buscapine, buscolysin, buscopan, butyiscopolamine, hyoscine N-butylbromide, N-butylscopolammonium bromide, scopolan bromide, butylscopolammonium bromide, N-butylscopolammonium chloride, hyoscine N-butylbromide, DD-234, hyoscine methiodide, hyoscine methobromide, methyiscopolamine nitrate, methylscopolammoium methyl sulfate, N-methylscine methyl sulfate, N-methyl scopolamine bromide, N-methyl scopolamine iodide, N-methyl scopolamine methylchloride, N-methyl scopolamine methyl sulfate, N-methyl scopolamine nitrate, skopyl, ulix bromide, N-methyl scopolamine, N-methylscopolamine methobromide, scopolamine methylchloride, N-methylscine methylsulfate, tematropium methylsulfate, and N-isopropylatropine, including salts and derivatives thereof.

L. Antioxidants

Antioxidants are chemicals that inhibit lipid oxidation. Some antioxidants (e.g., phenolic compounds) interrupt the free-radical chain of oxidative reactions by complexing with free radicals to form stable compounds that do not initiate or propagate further oxidation. Other antioxidants (e.g., acid compounds) slow the oxidative process by scavenging the reactive oxygen species. And still other antioxidants (e.g., chelators) slow oxidation by complexing with pro-oxidative metal ions.

Thousands of different types of antioxidants exist in nature. Some antioxidants of most importance to human health include without limitation astaxanthin, enzymes such as Superoxide Dismusase, vitamins A, C, and E, beta-carotene, selenium, lycopene, lutein, Coenzyme Q10, phytic acid, flavonoids, and polyphenols. Antioxidants are also separated into categories based upon whether they are water-soluble (hydrophilic) or fat-soluble (hydrophobic or lipophilic). Water-soluble antioxidants tend to predominantly react with oxidants in the cell cytosol and the blood plasma, while fat-soluble antioxidants tend to protect cell membranes from lipid peroxidation.

Various antioxidant compositions have been developed for the stabilization of oils and fats; most are mixtures of natural phenolic compounds (e.g., tocopherols) and acid compounds (e.g., ascorbic acid). While these antioxidant compositions inhibit lipid oxidation, they are not nearly as effective as synthetic phenolic antioxidants. One of the most effective antioxidants is ethoxyquin (6-ethoxy-1,2-dihydro-2,2,4-trimethylquinoline, sold under the trademark SANTOQUIN®), which is widely used as an antioxidant or preservative in feed supplements and a variety of other applications.

Several antioxidants are suitable for use in the compositions and methods of the present invention. The antioxidant may be a compound that interrupts the free-radical chain of oxidative reactions by protonating free radicals, thereby inactivating them. The antioxidant may be a compound that scavenges the reactive oxygen species. Alternatively, the antioxidant may be a compound that chelates the metal catalysts. The antioxidant may be a synthetic compound, a semi-synthetic compound, or a natural (or naturally-derived) compound.

In some aspects, the antioxidant is a substituted 1,2-dihydroquinoline. Substituted 1,2-dihydroquinoline compounds suitable for use in the invention generally correspond to Formula (I) as described in U.S. Patent App. Pub. No. US20080019860, particularly where the substituted 1,2-dihydroquinoline is 6-ethoxy-1,2-dihydro-2,2,4-trimethylquinoline (commonly known as ethoxyquin and sold under the trademark SANTOQUIN®) having the structure:

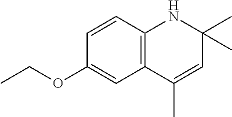 [SMILES: CCOc1ccc2c(c1)C(C)=CC(C)(C)N2]

In other aspects, the antioxidant includes, but is not limited to, ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, ice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, sinapic acid, succinic acid, stearal citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxy butyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat genii oil, zeaxanthin, or combinations thereof.

Further exemplary antioxidants include synthetic phenolic compounds, such as tertiary butyl hydroquinone (TBHQ); gallic acid derivatives, such as n-propyl gallate; vitamin C derivatives, such as ascorbyl palmitate; lecithin; and vitamin E compounds, such as, alpha-tocopherol.

M. Bioavailability Enhancing Agents

Bioavailability refers to the extent and rate at which the active moiety (drug or metabolite) enters systemic circulation, thereby accessing the site of action. Bioavailability for a given formulation provides an estimate of the relative fraction of the orally administered dose that is absorbed into the systemic circulation. Low bioavailability is most common with oral dosage forms of poorly water-soluble, slowly absorbed drugs. Insufficient time for absorption in the gastrointestinal tract is a common cause of low bioavailability. If the drug does not dissolve readily or cannot penetrate the epithelial membrane (e.g., if it is highly ionized and polar), time at the absorption site may be insufficient. Orally administered drugs must pass through the intestinal wall and then the portal circulation to the liver, both of which are common sites of first-pass metabolism (metabolism that occurs before a drug reaches systemic circulation). Thus, many drugs may be metabolized before adequate plasma concentrations are reached.

Bioavailability is usually assessed by determining the area under the plasma concentration-time curve (AUC). AUC is directly proportional to the total amount of unchanged drug that reaches systemic circulation. Plasma drug concentration increases with extent of absorption; the maximum (peak) plasma concentration is reached when drug elimination rate equals absorption rate. Peak time is the most widely used general index of absorption rate; the slower the absorption, the later the peak time.

The bioavailability of some drugs is increased when co-administered with food, particularly agents such as cannabinoids that are Class II drugs under the Biopharmaceutical Drug Classification System (Kelepu et al. (2013) Acta Pharmaceutica Sinica B 3:361-372; Amidon et al. (1995) Pharm. Res. 12:413-420; Charman et al. (1997) J. Pharm. Sci. 86:269-282; Winstanley et al. (1989) Br. J. Clin. Pharmacol. 28:621-628). It is the lipid component of the food that plays a key role in the absorption of lipophilic drugs and that leads to enhanced oral bioavailability (Hunt & Knox (1968) J. Physiol. 194:327-336; Kelepu et al. (2013) Acta Pharmaceutica Sinica B 3:361-372). This has been attributed to the ability of a high fat meal to stimulate biliary and pancreatic secretions, to decrease metabolism and efflux activity, to increase intestinal wall permeability, and to a prolongation of gastrointestinal tract (GIT) residence time and transport via the lymphatic system (Wagnera et al. (2001) Adv. Drug Del. Rev. 50:513-31; Kelepu et al. (2013) Acta Pharmaceutica Sinica B 3:361-372). High fat meals also elevate triglyceride-rich lipoproteins that associate with drug molecules and enhance intestinal lymphatic transport, which leads to changes in drug disposition and changes the kinetics of the pharmacological actions of poorly soluble drugs (Gershkovich et al. (2007) Eur. J. Pharm. Sci. 32:24-32; Kelepu et al. (2013) Acta Pharmaceutica Sinica B 3:361-372). However, co-administration of food with lipophilic drugs requires close control and/or monitoring of food intake when dosing such drugs, and can also be subject to problems with patient compliance (Kelepu et al. (2013) Acta Pharmaceutica Sinica B 3:361-372).

In some aspects, within the compositions and methods of the present invention, the bioavailability enhancing agent is an edible oil or fat comprising medium and/or long chain fatty acids. An edible oil is defined herein as an oil that is capable of undergoing de-esterification or hydrolysis in the presence of pancreatic lipase in vivo under normal physiological conditions. Specifically, digestible oils may be complete glycerol triesters of medium chain (C₇-C₁₃) or long chain (C₁₄-C₂₂) fatty acids with low molecular weight (up to C₆) mono-, di- or polyhydric alcohols. Medium and long chain fatty acids can comprise oleic acid, undecanoic acid, valeric acid, heptanoic acid, pelargonic acid, capric acid, lauric acid, and eicosapentaenoic acid.

Some examples of edible oils (also referred to as digestible oils) for use in this invention thus include: vegetable, nut, or seed oils (such as coconut oil, peanut oil, soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond, borage, peppermint and apricot kernel oils), and animal oils (such as fish liver oil, shark oil and mink oil).

In a further aspect, the bioavailability enhancing agent is substantially free of omega-6 fatty acids.

In other aspects, the bioavailability of the lipophilic active agent in a subject is at least about 1.5 times, 2 times, 2.5 times, 3 times, 3.5 times, 4 times, 4.5 times, 5 times, 5.5 times, 6 times, 6.5 times, 7 times, 7.5 times, 8 times, 8.5 times, 9 times, 9.5 times, 10 times greater, 10.5 times greater, 11 times greater, 11.5 times greater, 12 times greater, 12.5 times greater, 13 times greater, 13.5 times greater, 14 times greater, 14.5 times greater, 15 times greater, 15.5 times greater, 16 times greater, 16.5 times greater, 17 times greater, 17.5 times greater, 18 times greater, 18.5 times greater, 19 times greater, 19.5 times greater, or 20 times greater than the bioavailability of the lipophilic active agent in the subject in the absence of the bioavailability enhancing agent.

In a further aspect, the bioavailability of the lipophilic active agent in a subject is greater than 20% or at least about 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or greater.

Assays and methods for measuring lipophilic active agent bioavailability are well known in the art (see, e.g., Rocci & Jusko (1983) Comput. Programs Biomed. 16:203-215; Shargel & Yu (1999) Applied biopharmaceutics & pharmacokinetics (4th ed.). New York: McGraw-Hill; Hu & Li (2011) Oral Bioavailability: Basic Principles, Advanced Concepts, and Applications, John Wiley & Sons Ltd.; Karschner et al. (2011) Clinical Chemistry 57:66-75; Ohlsson et al. (1980) Clin. Pharmacol. Ther. 28:409-416; Ohlsson et al. (1982) Biomed. Environ. Mass Spectrom. 9:6-10; Ohlsson et al. (1986) Biomed. Environ. Mass Spectrom. 13:77-83; Karschner et al. (2010) Anal. Bioanal. Chem. 397:603-611).

N. Dosages and Concentrations

The active agents of the present invention are effective over a wide dosage range. For example, in treating adult humans, compositions and methods of the present invention comprise dosages of lipophilic active agents from 0.01 mg to 1,000 mg, from 0.5 mg to 500 mg, from 1 mg to 100 mg, from 5 mg to 50 mg, and from 10 mg to 25 mg. Alternatively, in treating adult humans, compositions and methods of the present invention comprise dosages of lipophilic active agents of 0.01 mg, 0.05 mg, 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg.

In other aspects, the concentration of lipophilic active agents within the compositions and methods of the present invention may range from 5 ppm to about 1000 ppm. In other embodiments, the concentration may range from about 50 to about 500 ppm. In still additional embodiments, the concentration may range from about 50 to about 200 ppm, particularly about 100 ppm.

In other aspects, the concentration of lipophilic active agents within the compositions and methods of the present invention may also be expressed as a percent of the active agent by weight. In one embodiment, the amount of the lipophilic active agent may range from about 0.0001% to about 20% by weight. In another embodiment, the amount may range from about of 1% to about 15% by weight. In yet another embodiment, the amount may range from 3.75% to about 10% by weight. In another embodiment, the amount may range from about 1% to about 99% by weight, from about 10% to about 80% by weight, and more typically, from about 20% to about 60% by weight. In another embodiment, the amount may be about 5% by weight, less than about 5% by weight, less than about 4% by weight, less than about 3% by weight, less than about 2% by weight, or less than about 1% by weight. In another embodiment, the amount may be greater than about 5%, greater than about 10%, greater than about 15%, greater than about 20%, greater than about 23%, greater than about 30%, greater than about 35%, greater than about 40%, greater than about 50%, greater than about 55%, greater than about 60%, greater than about 65%, greater than about 70%, or greater than about 75%.

In other aspects, the concentration of lipophilic active agents will vary depending on the total number of lipophilic active agents. For example, in compositions and methods of the present invention comprising more than one lipophilic active agent, the concentration of each lipophilic active may range from about 100 ppm to about 400 ppm (or from about 3.75% to about 30% by weight), with the total concentration of lipophilic active agents ranging from about 50 ppm to about 2000 ppm.

It is contemplated that the compositions of the present invention can include any ingredient (e.g., lipophilic active agent, carrier, etc.) or any combination thereof described throughout this specification. The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

O. Flavoring Agents

In some aspects, within the compositions and methods of the present invention, the flavoring agent is selected from the group consisting of vanilla, vanillin, ethyl vanillin, orange oil, peppermint oil, strawberry, raspberry, and mixtures thereof.

P. Lyophilization

Lyophilization, also known as freeze-drying, is a process whereby water is sublimed from a composition after it is frozen. The frozen solution is then typically subjected to a primary drying step in which the temperature is gradually raised under vacuum in a drying chamber to remove most of the water, and then to a secondary drying step typically at a higher temperature than employed in the primary drying step to remove the residual moisture in the lyophilized composition. The lyophilized composition is then appropriately sealed and stored for later use. Tang et al. (2004) Pharmaceutical Research 21:191-200 describes the scientific principles pertaining to freeze drying and guidelines for designing suitable freeze drying processes. Further description of freeze drying is found in Remington (2006) The Science and Practice of Pharmacy, 21^{st} edition, Lippincott Williams & Wilkins, pp. 828-831.

### II. Compositions

A. Pharmaceutical Compositions

In a further aspect, the lipophilic active agent administered within the methods of the present invention is formulated for oral administration, e.g. as a pharmaceutical composition. In some aspects, the pharmaceutical composition formulated for oral administration is formulated as a tablet, pill, capsule, liquid, gel, syrup, or slurry.

The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery. In a particular embodiment, the pharmaceutical composition is formulated for oral administration.

Active agents can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers, such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof, such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler, such as lactose, binders, such as starches, and/or lubricants, such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added. In some embodiments, the pharmaceutical composition is formulated for oral administration.

Additional components of the compositions of the present invention are understood by those of skill in the art and may generally be found in Remington, The Science and Practice of Pharmacy, Gennaro A. ed., p. 681-699, 20^{th} Edition, Lippincott, 2000.

B. Kits and Containers

Also contemplated are kits that include any one of the compositions disclosed throughout the specification and claims. In certain embodiments, the composition is comprised in a container. The container can be a bottle, dispenser, or package. The container can dispense a pre-determined amount of the composition. In certain aspects, the compositions is dispensed in a spray, dollop, or liquid. The container can include indicia on its surface. The indicia can be a word, an abbreviation, a picture, or a symbol.

### III. General Definitions

The pharmacokinetic profile of a drug is commonly described by the following parameters: Maximum plasma concentration (Cₘₐₓ), Time to maximum concentration (Tₘₐₓ), half-life (T_{1/2}), and Area under the curve (AUC). In other words, as used herein, AUC is the area under the curve of a graph of the concentration of a lipophilic active agent (typically plasma concentration) vs. time (i.e., measured from one time to another). “Cₘₐₓ” is defined as the measured concentration of the lipophilic active agent in the plasma at the point of maximum concentration. “Tₘₐₓ” is defined as the time to reach Cₘₐₓ. “T_{1/2}” is defined as the time for Cₘₐₓ to drop in half.

These parameters are to some extent interdependent, and are influenced in varying degrees by the outcome of interactions of food with drug dissolution, absorption, distribution, metabolism, and/or elimination. For example, for a certain drug, Cₘₐₓ might be mostly influenced by the rate of dissolution and absorption, while Tₘₐₓ might be mostly influenced by dissolution and distribution. For the same drug, T_{1/2} might be mostly influenced by metabolism and elimination, and AUC may be more or less influenced by all processes.

In most cases, changes in dissolution and absorption will have a significant impact on all parameters except perhaps on T_{1/2}. This implies that a formulation system, by which these two processes can be controlled and be made independent of food intake, will provide a more reliable and safer administration of the drug. Depending on the indication, the pharmacokinetic parameter that is most closely connected to therapeutic effect is either AUC, Cₘₐₓ, Tₘₐₓ or combinations thereof.

As used herein, “reducing the food effect” is defined as a less than about 1%, about 2%, about 3%, about 4%, about 5%, about 6%, about 7%, about 8%, about 9%, about 10%, about 15%, about 20%, about 25% or more difference in AUC, Cₘₐₓ, Tₘₐₓ or combinations thereof between subjects who had been fed or fasted prior to administration of a lipophilic active agent. In particular, “reducing the food effect” as used herein can refer to an almost equal AUC, Cₘₐₓ, and/or Tₘₐₓ between subjects who had been fed or fasted prior to administration of a lipophilic active agent, and in fact may refer to improved AUC, Cₘₐₓ, and/or Tₘₐₓ performance under fed conditions.

As used herein, the term “subject” treated by the presently disclosed methods in their many aspects is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disease, disorder, condition or the prophylactic diagnosis or treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some aspects, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular composition employed, the route of administration, the time of administration, the rate of excretion of the particular composition being employed, the duration of the treatment, other drugs, and/or materials used in combination with the particular composition employed, the age, sex, weight, condition, general health and prior medical history of the patient being treated, and like factors well known in the medical arts.

A physician having ordinary skill in the art can readily determine and prescribe the effective amount of the presently disclosed composition required. Accordingly, the dosage range for administration may be adjusted by the physician as necessary, as described more fully elsewhere herein.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

### EXAMPLES

### Example 1

### Background

A consumer study was undertaken with a primary objective of comparing speed of onset performance between a cannabis infused chocolate product formulation made using DEHYDRATECH™ technology and a concentration matched, control formulation made without DEHYDRATECH™ technology. As used herein, compositions incorporating DEHYDRATECH™ are compositions that incorporate a dehydrated mixture comprising a therapeutically effective amount of a lipophilic active agent and an edible oil comprising long chain fatty acids and/or medium chain fatty acids, particularly wherein dehydrated mixture is obtainable by the steps of:
i) combining a therapeutically effective amount of the lipophilic active agent with the edible oil comprising long chain fatty acids and/or medium chain fatty acids; and
ii) dehydrating the product of step (i), thereby producing the dehydrated mixture.

### Methodology

Three observation sessions were conducted under the direction of a third-party, contract research service provider at a professional research facility in San Francisco, Calif. Forty (40) respondents were recruited to achieve a minimum participation of 30. The study was designed as a longitudinal panel so that the same respondents participated across all study days, to the extent possible, where there was a total of three study days.

This was a blind study; the Test (DEHYDRATECH™) and Control product identities were not disclosed to the respondents, nor to the study coordinators. Respondents were paid an incentive for their participation.

Respondents were recruited based on the following criteria:
Have an active/legal license for medical marijuana in California;
Age 21 to 64;
Regularly use edible cannabis products and comfortable consuming a dosage of 10 mg;
Do not have diabetes, lactose intolerance or allergies related to chocolate
Have a mobile device; and
Articulate.

Respondents were also instructed to follow these protocols in order to participate:
Do not use cannabis or other psychoactive agents for 24 hours prior to each session;
Day 1—fast after midnight prior to the session; and
Days 2 and 3—eat breakfast and consume a gluten free chocolate chip cookie 30 minutes prior to the product.

Two products were tested, one identified as “Green” and one identified as “Blue”. Respondents were evenly split into two groups by product; each group gathered in a separate room. The product that each respondent tested was alternated by session (for example, those who tested Green on day 1 then tested Blue on day 2 and switched back to Green on day 3).

Product feedback was gathered via online surveys completed at the end of each session, aided by handouts that were used to track specific experience times throughout the session in real-time. The product test consisted of two chocolates per respondent per session, of the same version. Each chocolate had 5 mg of tetrahydrocannabinol (THC), for total individual doses of 10 mg per study day.

The Green product was the Test version with the DEHYDRATECH™ technology. The Blue product was the Control version without the DEHYDRATECH™ technology.

The sessions were designed as follows:
1. Respondents checked-in, confirmed identity, medical marijuana card, key qualifications, and signed an informed consent agreement;
2. Respondents proceeded to designated session room at 9 am (local Pacific time) for kickoff and ingested product at 9:30 am;
3. Respondents tracked key time elements throughout the session using a structured handout—Study Coordinator reminded respondents to track time accurately roughly every 15 minutes;
4. Respondents remained in the session throughout the product experience, receiving snacks, lunch and refreshments commencing at 11 am (local Pacific time) as described below; and
5. Once product experience had ended, respondents completed exit survey online and checked-out.

The following table provides an overview of the respondent make up and treatment per session.

TABLE 1
Respondent Make Up and Treatment Per Session

| Day 1 - Tuesday Oct. 3, 2017 | Day 2 - Thursday Oct. 5, 2017 | Day 3 - Monday Oct. 9, 2017 |
|---|---|---|
| 20 Green respondents | 19 Green respondents | 18 Green (due to no shows) |
| 19 Blue respondents | 20 Blue respondents | 20 Blue respondents |
| — | Same respondents as Day 1 | 7 dropped & 7 new respondents |
| Respondents fasted | Respondents ate breakfast and a | Respondents ate breakfast and a |
| | gluten free chocolate chip | gluten free chocolate chip |
| | cookie | cookie |
| Snacks at 11 am, | Snacks at 11 am, | Snacks at 11 am, |
| lunch just before 12:30 pm | lunch at 12 pm | lunch at 12 pm |

### Results

Respondents recorded the time they consumed the product and the time that they began to feel effects of the product. Onset performance was calculated by subtracting the effect start time from the consumption time.

In total, there were 39 respondents that sampled both a green (DEHYDRATECH™) test product and a blue (Control) product on at least two separate days of the study. Of these, there were 22 instances (56%) where the respondents rated the DEHYDRATECH™ formulation as faster acting than the Control formulation. Furthermore, of the instances where the DEHYDRATECH™ formulation outperformed the Control, it did so with a median performance improvement that was more pronounced in light vs. moderate vs. heavy users as follows:

Light Users: 50% Faster

Moderate Users: 28% Faster

Heavy Users: 26% Faster

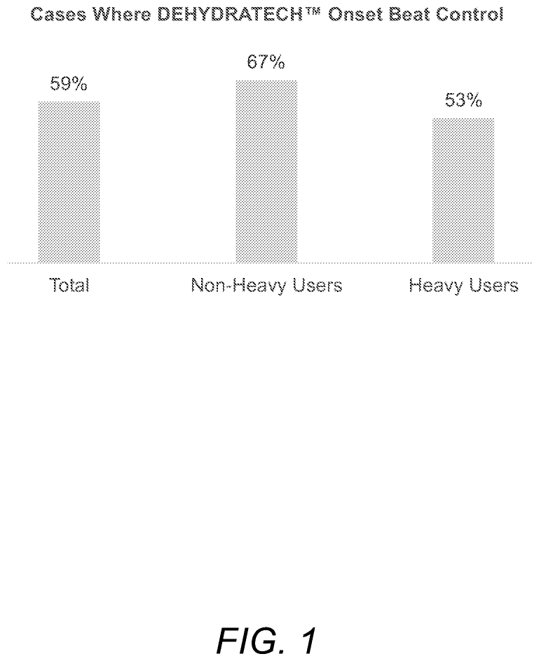

In 29 cases where the DEHYDRATECH™ test and Control products were directly compared over day 2 and day 3 (with food), 59% indicated that the DEHYDRATECH™ version had faster onset performance than the Control version (FIG. 1).

Product performance was further observed by cannabis usage levels. Heavy users report that they use cannabis more than 10 times monthly; Non-Heavy users report using cannabis 10 or fewer times monthly. Just over half of all respondents (52%) were classified as heavy users.

Among the 29 cases where DEHYDRATECH™ test and Control products were directly compared over days 2 and 3 (with food), 67% of non-heavy users indicated the DEHYDRATECH™ version had faster onset performance versus 53% of heavy users (FIG. 1).

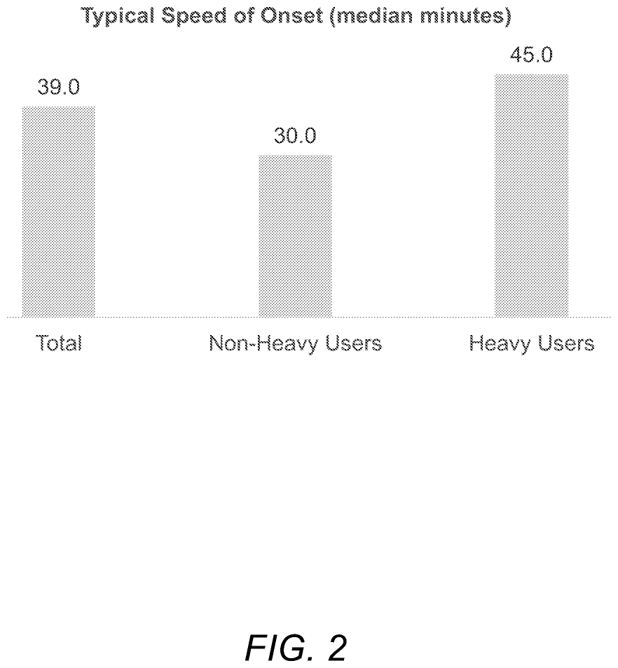

Respondents were asked about their typical experience with edible onset performance apart from the study, supporting wide variances known in the industry. The median result was 39 minutes to feel effects after taking cannabis edibles in general. Non-heavy users indicated faster speed of onset, with a median of 30 minutes, compared to slower onset for heavy users, with a median of 45 minutes (FIG. 2).

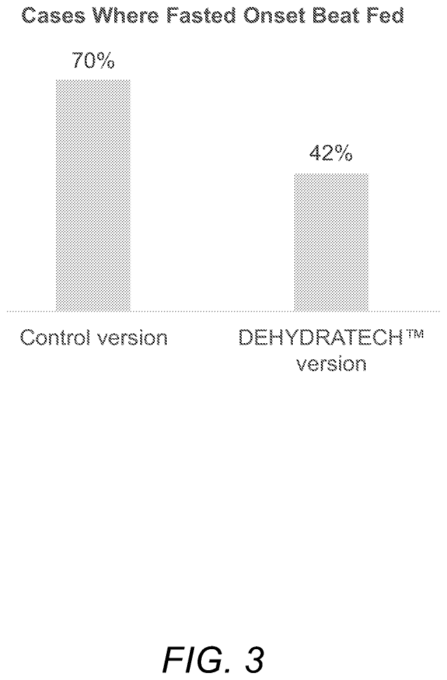

Eating edibles on an empty stomach generally makes the effects quicker and harder, due to the digestion and absorption process of THC. This was observed for the Control version, such that of the 10 instances where respondents sampled the Control on Day 1 (fasted) and Day 3 (fed) and experienced faster onset time with the Control version than with the DEHYDRATECH™ version, 70% of these instances indicated the fastest Control version performance after fasting. The DEHYDRATECH™ version, however, appeared less impacted by food; where faster onset speeds were split fairly evenly 58/42 between those with and without food, among 12 observations where respondents sampled the DEHYDRATECH™ version on Day 1 (fasted) and Day 3 (fed) and experienced faster onset time with the DEHYDRATECH™ version than with the Control version (FIG. 3).

### Discussion

As described above, the study results indicated that the DEHYDRATECH™ formulation acted faster in the majority of cases compared to the Control formulation. However, speed of onset performance times varied across the respondents, based on which evaluations for purposes of this report were generally made at the intra-subject respondent level rather than on an inter-subject product type level.

This study also generally indicated that the effects of the DEHYDRATECH™ formulation were more pronounced in non-heavy users than in heavy users. Non-heavy users appear to be more receptive to the relatively low 5 mg THC dosage level studied here overall and to the effects of the DEHYDRATECH™ technology in particular. This finding was suggestive of a putative mode of action of the DEHYDRATECH™ technology in enhancing intestinal bioabsorption, whereby long chain fatty acids and/or medium chain fatty acids that are associated with the lipophilic active substance of interest (in this case THC) through DEHYDRATECH™'s processing methodology may stimulate intestinal bioabsorption via biliary interaction and lymphatic uptake rather than the hepatic uptake and biotransformation independent of biliary complexing that conventional THC edibles are thought to rely upon. Based on this, the DEHYDRATECH™ technology may initially stimulate faster but less pronounced effects of delta-9 THC without liver biotransformation, whereas conventional THC edibles instead result in slower but more pronounced effects of 11-OH THC. Accordingly, heavy users may be less sensitive to the effects of such 5 mg “low dose” products and further may overlook the milder initial effects of the DEHYDRATECH™ technology; where they may be more accustomed to the more pronounced effects of 11-OH THC that is common with conventional edibles.

This study also provided an opportunity to assess performance findings after fasting compared to performance findings after allowing subjects to consume a meal and also ingest a light snack prior to test article dosing. Generally speaking, fasting versus fed pre-study conditions impact bioabsorption of ingested agents, which is why much of the analyses provided above focus on Days 2 and 3 of the study (fed conditions) where this study gathered more data than could be amassed from Day 1 of the study (fasted conditions). Not surprisingly, as described above, fasting pre-study conditions resulted in faster speed of onset for the Control formulation in 70% of cases where the Control formulation was sampled on Day 1 and Day 3 compared to the DEHYDRATECH™ formulation sampled on Day 2. However, it was intriguing to observe that in those instances where the DEHYDRATECH™ formulation was sampled on Day 1 and Day 3 compared to the Control formulation sampled on Day 2, the DEHYDRATECH™ formulation acted faster than the Control in almost equal proportions (i.e., 58/42) irrespective of whether the respondents were fed or fasted respectively prior to the study. This finding may additionally relate to a putative mode of action of the DEHYDRATECH™ technology in enhancing intestinal bioabsorption as described above, whereby the onset of THC effectiveness upon fed conditions with the DEHYDRATECH™ formulation may actually be enhanced rather than delayed by the presence of foodstuffs in the intestine due to stimulation thereby of the release of bile and, in turn, induction of absorption preferentially compared to conventional formulations lacking the DEHYDRATECH™ process. Indeed, the findings from this study seemed to indicate that instances where the DEHYDRATECH™ formulation acted faster than the Control formulation were slightly more evident after fed pre-study conditions as an unconventional outcome with cannabis edibles in general, whereas those instances where the Control formulation acted faster than the DEHYDRATECH™ formulation were most pronounced after fasted pre-study conditions as one would expect.

1.-50. (canceled)

51. A composition for reducing the food effect of a lipophilic active agent, comprising:
i) a lipophilic active agent selected from the group consisting of cannabinoids, terpenes and terpenoids, non-steroidal anti-inflammatory drugs (NSAIDs), vitamins, nicotine, phosphodiesterase 5 (PDE5) inhibitors, Maca extract, hormones, fentanyl, buprenorphine, scopolamine, and antioxidants;
ii) an emulsifier; and
iii) a bioavailability enhancing agent.

52. The composition according to claim 51, wherein the bioavailability enhancing agent bioavailability enhancing agent is a protective colloid, an edible oil or fat.

53. The composition according to claim 52, wherein the edible oil is coconut oil, peanut oil, soybean oil, safflower seed oil, corn oil, olive oil, castor oil, cottonseed oil, arachis oil, or sunflower seed oil.

54. The composition according to claim 53, wherein the edible oil comprises fatty acids selected from the group consisting of oleic acid, undecanoic acid, valeric acid, heptanoic acid, pelargonic acid, capric acid, lauric acid, and eicosapentaenoic acid.

55. The composition according to claim 51, further comprising an edible substrate selected from the group consisting of tea leaves, coffee beans, cocoa powder, meats, fish, fruits, vegetables, dairy products, legumes, pastas, breads, grains, seeds, nuts, spices, and herbs.

56. A process for reducing food effect in a lipophilic active agent infused food product comprising the steps of:
(a) contacting a food product with an oil comprising a lipophilic active agent and a bioavailability enhancing agent; and
(b) dehydrating the food product;
thereby producing a lipophilic active agent food product with reduced food effect;
wherein the lipophilic active agent food product with reduced food effect comprises a therapeutically effective amount of the lipophilic active agent, and further wherein the bioavailability enhancing agent comprises an edible oil comprising long chain fatty acids and/or medium chain fatty acids; and further wherein:
(i) the lipophilic active agent is selected from the group consisting of cannabinoids, terpenes and terpenoids, non-steroidal anti-inflammatory drugs (NSAIDs), vitamins, nicotine, phosphodiesterase 5 (PDE5) inhibitors, Maca extract, hormones, fentanyl, buprenorphine, scopolamine, and antioxidants; and
(ii) the food product is selected from the group consisting of tea leaves, coffee beans, cocoa powder, meats, fish, fruits, vegetables, dairy products, legumes, pastas, breads, grains, seeds, nuts, spices, and herbs.

57. The process according to claim 56, wherein step (b) further comprises contacting the food product with a starch.

58. The process according to claim 57, wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

59. The process according to claim 56, further comprising an edible oil selected from the group consisting of coconut oil, peanut oil, soybean oil, safflower seed oil, corn oil, olive oil, castor oil, cottonseed oil, arachis oil, or sunflower seed oil.

60. The process according to claim 56, wherein the bioavailability of the lipophilic active agent in a subject is at least 2 times greater than the bioavailability of the lipophilic active agent in the subject in the absence of the edible oil comprising long chain fatty acids and/or medium chain fatty acids.

61. The process according to claim 56, wherein the bioavailability of the lipophilic active agent in a subject is at least 5 times greater than the bioavailability of the lipophilic active agent in the subject in the absence of the edible oil comprising long chain fatty acids and/or medium chain fatty acids.

62. The process according to claim 56, wherein the bioavailability of the lipophilic active agent in a subject is at least 10 times greater than the bioavailability of the lipophilic active agent in the subject in the absence of the edible oil comprising long chain fatty acids and/or medium chain fatty acids.

63. The process according to claim 56, wherein the edible oil comprising long chain fatty acids and/or medium chain fatty acids is substantially free of omega-6 fatty acids.

64. The process according to claim 56, further comprising the step of steeping the lipophilic active agent infused tea leaves, coffee beans, or cocoa powder in a liquid, thereby producing the lipophilic active agent infused beverage product with reduced food effect.

65. A process for reducing food effect in a ready-to-drink beverage composition comprising a lipophilic active agent, obtainable by the steps of:
(a) contacting an emulsifier with an oil comprising a lipophilic active agent and a bioavailability enhancing agent, thereby producing a mixture comprising the emulsifier, the oil comprising the lipophilic active agent, and the bioavailability enhancing agent;
(b) dehydrating the mixture, thereby producing a dehydrated mixture comprising the emulsifier, the oil comprising the lipophilic active agent, and the bioavailability enhancing agent; and
(c) combining the dehydrated mixture with a ready-to-drink beverage composition, thereby producing a ready-to-drink beverage composition comprising the lipophilic active agent with reduced food effect;
wherein:
(i) the bioavailability enhancing agent comprises an edible oil comprising long chain fatty acids and/or medium chain fatty acids;
(ii) the ready-to-drink beverage composition comprising a lipophilic active agent comprises a therapeutically effective amount of the lipophilic active agent; and
(iii) the lipophilic active agent is selected from the group consisting of cannabinoids, terpenes and terpenoids, non-steroidal anti-inflammatory drugs (NSAIDs), vitamins, nicotine, phosphodiesterase 5 (PDE5) inhibitors, Maca extract, hormones, fentanyl, buprenorphine, scopolamine, and antioxidants.

66. The process according to claim 55, wherein the emulsifier is selected from the group consisting of inulin, gum arabic, modified starch, pectin, xanthan gum, gum ghatti, gum tragacanth, fenugreek gum, mesquite gum, mono-glycerides and di-glycerides of long chain fatty acids and/or medium chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, and combinations thereof.

67. The process according to claim 55, wherein step (b) further comprises contacting the food product with a starch.

68. The process according to claim 57, wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

69. The process according to claim 56, wherein the bioavailability of the lipophilic active agent in a subject is at least 2 times greater than the bioavailability of the lipophilic active agent in the subject in the absence of the edible oil comprising long chain fatty acids and/or medium chain fatty acids.

70. The process according to claim 56, wherein the bioavailability of the lipophilic active agent in a subject is at least 5 times greater than the bioavailability of the lipophilic active agent in the subject in the absence of the edible oil comprising long chain fatty acids and/or medium chain fatty acids.

71. The process according to claim 56, wherein the bioavailability of the lipophilic active agent in a subject is at least 10 times greater than the bioavailability of the lipophilic active agent in the subject in the absence of the edible oil comprising long chain fatty acids and/or medium chain fatty acids.
